(19) United States
(12) Patent Application Publication
(10) Pub. No.: US 2026/0210838 A1
(43) Pub. Date: Jul. 23, 2026

(54) DEVICES AND METHODS FOR OPTICAL ANALYSIS OF BIOLOGICAL SAMPLES

(71) Applicant: VITAL BIOSCIENCES INC., Mississauga (CA)

(72) Inventors: Akhil RAJAGOPAL KOZHIPURAM, Brampton (CA); Wisam ALFAQHERI, London (CA); Miguel BARREIROS, Lisboa (PT); Mark Ronald BREMER, Cambridge (CA); Todd CAMPBELL, Holliston, MA (US); Erden ERTORER, Niagara Falls (CA); Farnoud KAZEMZADEH, Waterloo (CA); Mounir KOUSSA, Oakville (CA); Taehyung Chris LEE, Oakville (CA); Mate LENGYEL, MISSISSAUGA (CA); Eric James MAHONEY, Hamilton (CA); Abdolreza MOVAGHAR, San Diego, CA (US); Claire Grace NOEL, King City (CA); Paul Said Ehrlich PEREZ, Toronto (CA); Sergey SANDERS, Cambridge (CA)

(21) Appl. No.: 19/451,523

(22) Filed: Jan. 16, 2026

Related U.S. Application Data
(63) Continuation of application No. PCT/IB2024/057073, filed on Jul. 21, 2024.
(60) Provisional application No. 63/514,965, filed on Jul. 21, 2023.

(51) Int. Cl. G01N 21/01; G01N 21/77

(52) U.S. Cl. CPC G01N 21/01; G01N 21/77

(57) ABSTRACT

Devices and methods for determining analytes in biological samples are disclosed. A device includes a motor to rotate a disc having one or more wells at a common radius. Each well contains one or more reagents and is configured to receive an aliquot of a fluid comprising one or more components of a biological sample. The device also includes one or more illumination sources and one or more detectors. During a detection period while the disc is spinning, each illumination source illuminates each well, and each detector detects one or more optical signals from each well and produces at least one reading for each well per revolution of the disc to facilitate determination of one or more analytes in the biological sample.

### CROSS-REFERENCE TO RELATED APPLICATIONS

This application claims priority to and is a continuation of International Application No. PCT/IB2024/057073 filed Jul. 21, 2024, which claims the benefit of U.S. Provisional Patent Application No. 63/514,965 filed Jul. 21, 2023, entitled “DEVICES AND METHODS FOR OPTICAL ANALYSIS OF BIOLOGICAL SAMPLES”, which are incorporated by reference herein in their entirety.

### TECHNICAL FIELD

The present disclosure relates to devices and methods that allow running reactions and analyzation of biological samples in microfluidic devices.

### BACKGROUND

Currently, 70% of all medical decisions rely on lab-based diagnostics but today, the diagnostic process is disjointed from how care is delivered. The primary care system requires patients to travel to external phlebotomy sites to draw blood, which is sent to labs via courier, and processed overnight. This means that lab results reach health care professionals long after the patient has left. This friction in care delivery and disease management leads to tremendous waste in the healthcare system:
a. Patients often delay getting lab tests or fail to adhere to lab testing, or subsequent care recommendations.
b. The gap in the diagnostic process leads to missed tests, missed diagnosis, a lack of intervention, and ultimately poor outcomes.
c. Health care professionals waste time tracing lab orders to patient encounter notes. When intervention is needed, more time is wasted in reaching out to patients and driving subsequent steps in the patient's care pathway.

These problems are even more acute when caring for rural populations or patients belonging to groups facing adverse social determinants of health, where there are many challenges in ensuring successful follow ups from an initial patient encounter.

Several companies have built point-of-care instruments to bridge this divide. However, these instruments are limited to single types of tests and fail to completely meet the workflow needs of primary care providers for a single system that produces simple, comprehensive, and fast test results. A product to meet these needs is currently under development. It achieves this through a highly automated workflow enabled through the use of microfluidics.

Moreover, different reactions may last over very different time periods. For instance, one endpoint assay may take less than a few minutes to run and the other may take more than ten minutes to run. In many conventional settings, one has to synchronize all the chemistries to finish at the same time and then perform the measurement. Further, different assays may rely on different wavelengths for illumination or detection. For a large number of assays, this may take a significant amount of time and resource and in some cases may not be practical.

Accordingly, there remains a need for improved discs, devices and methods for performing a large number of reactions and analyzation in parallel using microfluidics technology, allowing for user-friendly and point-of-care deployment of such technology.

### SUMMARY

The present disclosure addresses these and other needs in the art by providing discs, devices and methods that can perform measurement while the discs are spinning and that can provide uniform heating or cooling to the discs without the use of fans.

The discs, devices, and methods of the present disclosure have other features and advantages that will be apparent from, or are set forth in more detail in, the accompanying drawings, which are incorporated herein, and the following Detailed Description, which together serve to explain certain principles of exemplary embodiments of the present disclosure.

In certain embodiments, a device for determining one or more analytes in a biological sample, the device comprising: a motor configured to rotate a disc having one or more first wells at a first common radius relative to a rotational axis of the disc, wherein each of the one or more first wells contains one or more reagents disposed therein and is configured to receive an aliquot of a fluid comprising one or more components of the biological sample; one or more illumination sources configured to illuminate the one or more first wells during a detection period while the motor is rotating the disc; and one or more detectors configured to detect one or more optical signals from the one or more first wells during the detection period while the motor is rotating the disc, wherein each of the one or more detectors produces at least one reading for each of the one or more first wells per revolution of the disc during the detection period, thereby facilitating determination of the one or more analytes in the biological sample.

In certain embodiments, the biological sample comprises blood. In certain embodiments, the one or more analytes comprise one or more of hemoglobin A1C (HbA1C), ketones, aspartate transaminase (AST), alanine transaminase (ALT), lactate, gamma-glutamyltransferase (GGT), glucose, calcium, high-density lipoprotein (HDL), low-density lipoprotein (LDL), sodium ion (Na+), potassium ion (K+), chloride ion (Cl−), total cholesterol, alkaline phosphatase (ALP), total carbon dioxide (tCO2), total protein, lactate dehydrogenase (LDH), blood urea nitrogen (BUN), direct bilirubin (dBil), total bilirubin (tBil), creatine kinase (CK), creatinine, triglycerides, albumin, and hemolysis, icterus, lipemia (HIL).

In certain embodiments, the one or more reagents comprise one or more lyophilized beads. In certain embodiments, the one or more first wells comprise a plurality of first wells. In certain embodiments, the plurality of first wells comprises at least five first wells, at least ten first wells, at least fifteen first wells, at least twenty first wells, at least twenty five first wells, at least thirty first wells, at least forty first wells, or at least fifty first wells. In certain embodiments, at least some of the plurality of first wells contain a same amount of the one or more components of the biological sample. In certain embodiments, at least some of the plurality of first wells contain different amounts of the one or more components of the biological sample. In certain embodiments, at least some of the plurality of first wells receive the fluid comprising a same dilution buffer. In certain embodiments, at least one of the plurality of first wells receive the fluid comprising the same dilution buffer at a different factor. In certain embodiments, at least some of the plurality of first wells receive the fluid comprising different buffers.

In certain embodiments, the disc is divided into a plurality of slices. In certain embodiments, at least one of the plurality of slices comprises at least one first well. In certain embodiments, the disc further comprises at least one second well positioned at a second common radius relative to the rotational axis of the disc. In certain embodiments, the second common radius is shorter than the first common radius.

In certain embodiments, the one or more illumination sources provide illumination at one or more wavelengths from about 280 nm to about 1000 nm. In certain embodiments, the one or more illumination sources comprise a plurality of illumination sources, each providing illumination at a corresponding wavelength in the plurality of wavelengths. In certain embodiments, each of the plurality of illumination sources is positioned at the first common radius relative to the rotational axis of the disc.

In certain embodiments, for each of the one or more first wells, each of the one or more detectors produces a plurality of readings per revolution of the disc during the detection period. In certain embodiments, the one or more detectors comprises a plurality of detectors, each configured to detect an optical signal indicative of a corresponding wavelength in the plurality of wavelengths. In certain embodiments, each of the plurality of detectors comprises a filter and a receiver. In certain embodiments, each of the plurality of detectors is positioned at the first common radius relative to the rotational axis of the disc.

In certain embodiments, the device further comprises: a first plate for housing the disc; a first assembly disposed at a first side of the first plate and comprising the plurality of illumination sources; a second assembly disposed at a second side of the first plate and comprising the plurality of detectors; a third assembly disposed at or adjacent to the first plate and configured for regulating a temperature of the biological sample or the fluid comprising the one or more components of the biological sample in the disc; a fourth assembly disposed at or adjacent to the first plate and configured for determining a location of the disc relative to a home location; an adapter plate configured for mounting the device to an instrument; or any combination thereof.

In certain embodiments, the first, second, third or fourth assembly comprises a printed circuit board. In certain embodiments, the second assembly comprises a second plate disposed at a second side of the first plate and configured for mounting the plurality of detectors.

In certain embodiments, the disc is rotatable relative to the third assembly; and the third assembly comprises a radiation heat source configured for maintaining the biological sample or the fluid comprising the one or more components of the biological sample at a desired temperature.

In certain embodiments, the third assembly is disposed below the disc. In certain embodiments, rotation of the disc creates air circulation between the disc and the radiation heat source, thereby facilitating uniform heating or cooling of the disc by the radiation heat source. In certain embodiments, the home location corresponds to the one or more illumination sources, the one or more detectors, the motor, or any combination thereof.

In certain embodiments, the device further comprises: a spindle, wherein the disc is disposed on the spindle; and a locking mechanism minimizing movement of the disc relative to the spindle, the locking mechanism comprising: a locking element having a first end and a second end; a collar extending radially from the first end; and a disc lock disposed on the second end, wherein the disc is received on the locking element between the collar and the disc lock.

In certain embodiments, a method for determining one or more analytes in a biological sample, the method comprising: A) operating the motor of the device of claim 1 to rotate a disc disposed in the device, wherein the disc comprises one or more first wells at a first common radius relative to a rotational axis of the disc, and one or more of the first wells contains one or more reagents disposed therein and an aliquot of a fluid comprising one or more components of the biological sample; B) illuminating, using the one or more illumination sources of the device, the one or more first wells during a detection period while the motor is rotating the disc; C) detecting, using the one or more detectors of the device, one or more optical signals from the one or more first wells during the detection period while the motor is rotating the disc, wherein each of the one or more detectors produces at least one reading for each of the one or more first wells per revolution of the disc during the detection period; and D) determining, based on the at least one reading for each of the one or more first wells per revolution of the disc during the detection period, the one or more analytes in the biological sample.

In certain embodiments, in the detecting C), each of the one or more detectors produces a plurality of readings for each of the one or more first wells per revolution of the disc during the detection period; and the determining D) comprises averaging the plurality of readings for each of the one or more first wells per revolution of the disc during the detection period. In certain embodiments, for each of the one or more first wells per revolution of the disc during the detection period, the detecting C) detects light transmitted through each of the one or more first wells, thereby producing the at least one reading that is indicative of light absorption by each of the one or more first wells.

In certain embodiments, the detection period comprises a first detection period; and the method further comprises: E) rotating, prior to the first detection period, the disc according to a first predefined profile to promote mixing of the aliquot of the fluid with the one or more reagents disposed in each of the one or more first wells. In certain embodiments, the first predefined profile comprises alternately accelerating and decelerating rotation of the disc within a first speed range.

In certain embodiments, the disc further comprises at least one second well positioned at a second common radius relative to the rotational axis of the disc, wherein the second common radius is shorter than the first common radius; the detection period comprises a second detection period; and the method further comprises: F) rotating, subsequent to the first detection period and prior to the second detection period, the disc according to a second predefined profile to promote mixing of the aliquot of the fluid with the one or more reagents disposed in at least one second well. In certain embodiments, the second predefined profile comprises alternately accelerating and decelerating rotation of the disc within a second speed range. In certain embodiments, the one or more first wells comprise a plurality of first wells spaced apart circumferentially from each other.

In certain embodiments, the detecting C) comprises detecting, using the one or more detectors, one or more optical signals from each spacing between adjacent first wells in the plurality of first wells during the detection period while the motor is rotating the disc, wherein each of the one or more detectors produces at least one reading for each spacing between adjacent first wells in the plurality of first wells per revolution of the disc during the detection period.

In certain embodiments, the one or more illumination sources provide illumination at a plurality of wavelengths, wherein each of the plurality of wavelengths corresponds to one or more respective analytes; and the one or more detectors comprises a plurality of detectors, each configured to detect an optical signal indicative of the one or more analytes at the corresponding wavelength.

In certain embodiments, the method further comprises: G) monitoring the at least one reading produced by each of the plurality of detectors for each of the plurality of first wells per revolution of the disc during the detection period; and H) determining, based on the monitoring G), whether the at least one reading produced by each of the plurality of detectors for each of the plurality of first wells per revolution of the disc during the detection period is normal or abnormal. In certain embodiments, the method further comprises: I) adjusting or terminating the method if it is determined that abnormality occurs.

### BRIEF DESCRIPTION OF THE DRAWINGS

The foregoing summary, as well as the accompanying drawings, which are incorporated into and constitute a part of this specification, illustrate one or more exemplary embodiments of the present disclosure and, together with the Detailed Description, serve to explain the principles and implementations of exemplary embodiments of the invention. The accompanying drawings are not necessarily to scale. The specific design features of the present invention as disclosed herein, including, for example, specific dimensions, orientations, locations, and shapes will be determined in part by the particular intended application and use environment. In addition, the components illustrated in the figures are combinable in any useful number and combination.

In the drawings:

FIG. 1A is a perspective view schematically illustrating a device in accordance with some exemplary embodiments of the present disclosure;

FIG. 1B is an exploded view schematically illustrating the device of FIG. 1A in accordance with some exemplary embodiments of the present disclosure;

FIG. 1C is a perspective view schematically illustrating the device of FIG. 1A receiving a disc in accordance with some exemplary embodiments of the present disclosure;

FIGS. 2A, 2B, 2C and 2D are schematic diagrams illustrating optical measurements in accordance with some exemplary embodiments of the present disclosure;

FIGS. 3A, 3B and 3C are schematic diagrams illustrating effects of tilting on optical measurements in accordance with some exemplary embodiments of the present disclosure;

FIG. 4A is a schematic diagram illustrating a disc in accordance with some exemplary embodiments of the present disclosure;

FIG. 4B is a schematic diagram illustrating light absorption through some structures of the disc of FIG. 4A in accordance with some exemplary embodiments of the present disclosure;

FIG. 4C is a schematic diagram illustrating a portion of the disc of FIG. 4A in accordance with some exemplary embodiments of the present disclosure;

FIG. 5 is a schematic diagram illustrating another disc in accordance with some exemplary embodiments of the present disclosure;

FIG. 6 is a schematic diagram illustrating a process in accordance with some exemplary embodiments of the present disclosure;

FIGS. 7A, 7B, 7C, 7D, 7E, 7F, 7G, 7H, 7I, 7J, 7K and 7L are schematic diagrams collectively illustrating a process in accordance with some exemplary embodiments of the present disclosure;

FIG. 8 is a flowchart illustrating a method in accordance with some exemplary embodiments of the present disclosure;

FIGS. 9A and 9B are flowcharts collectively illustrating another method in accordance with some exemplary embodiments of the present disclosure;

FIG. 10 is a table listing some analytes and corresponding parameters in accordance with some exemplary embodiments of the present disclosure;

FIG. 11A is a top perspective view illustrating a spindle of the device of FIG. 1B in accordance with some exemplary embodiments of the present disclosure;

FIG. 11B is a side cross-section view illustrating the spindle of FIG. 11A;

FIG. 12A is a first side view illustrating a disc in accordance with some exemplary embodiments of the present disclosure; and

FIG. 12B is a second side view illustrating the disc of FIG. 12A.

### DETAILED DESCRIPTION

The present disclosure provides discs, devices, and methods for determining analytes by measuring optical signals while the discs are spinning. In various embodiments, the discs, devices, and methods of the present disclosure are configured to allow for running a large number of reactions in parallel, and for optical detection and data acquisition for all chemical reactions, either kinetic reaction (e.g., measuring the slope) or endpoint reaction (e.g., waiting for the reaction to reach the plateau). In some embodiments, the discs, devices, and methods of the present disclosure are also configured to allow for optical measurement at each of multiple wavelengths and every revolution while the disc is spinning. In some embodiments, the discs, devices, and methods of the present disclosure are configured to allow for spatial averaging (e.g., within a well) and temporal averaging (e.g., across multiple revolutions). In some embodiments, the discs, devices, and methods of the present disclosure are configured to enable cooling of the discs without the use of fans.

### Exemplary Devices

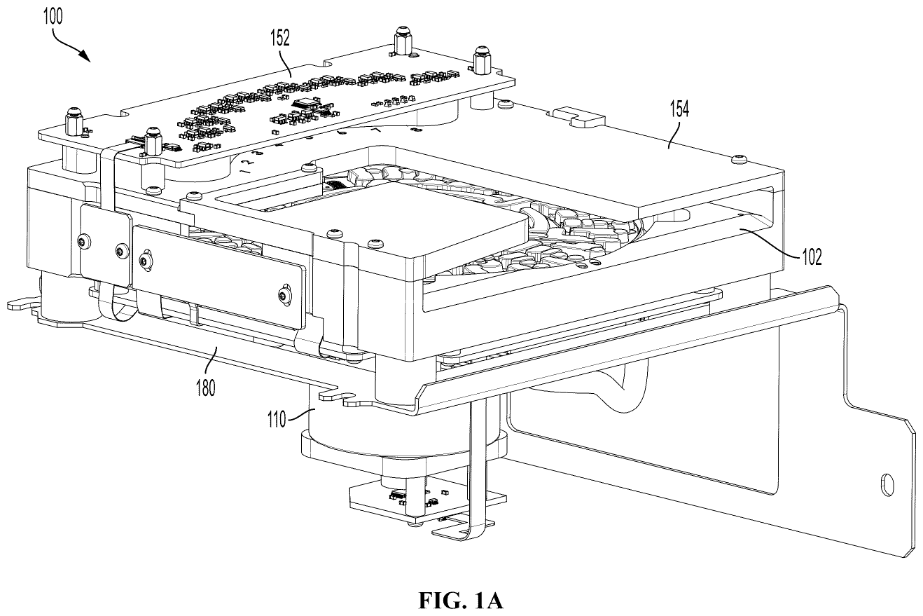

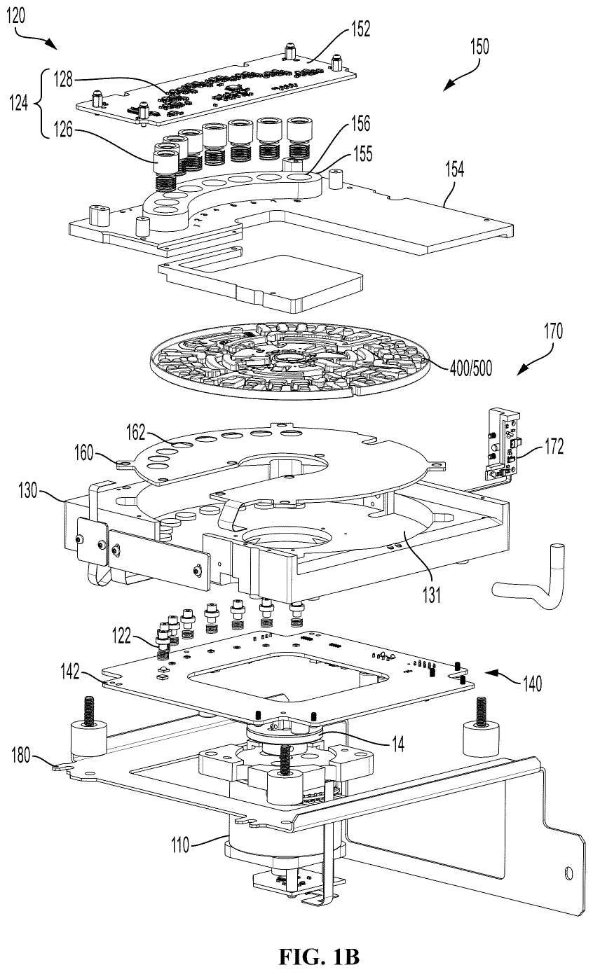

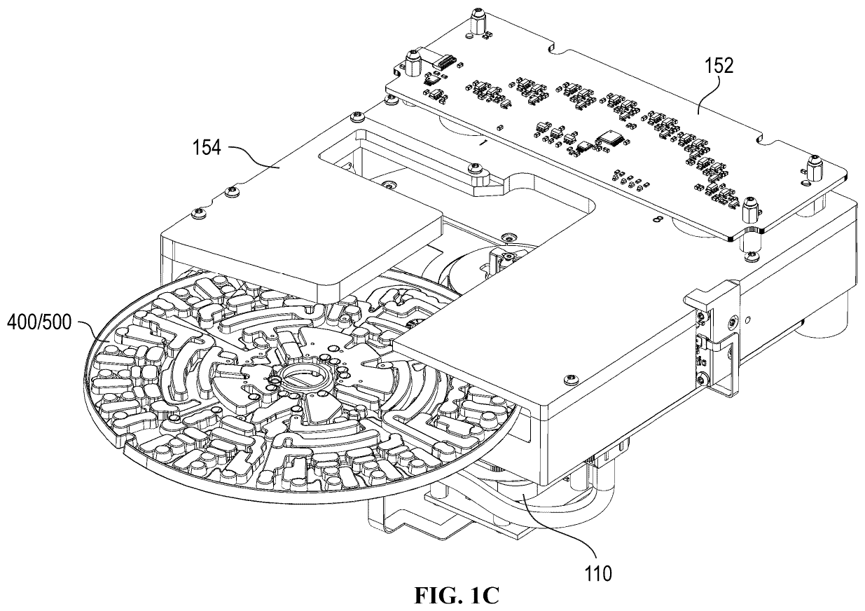

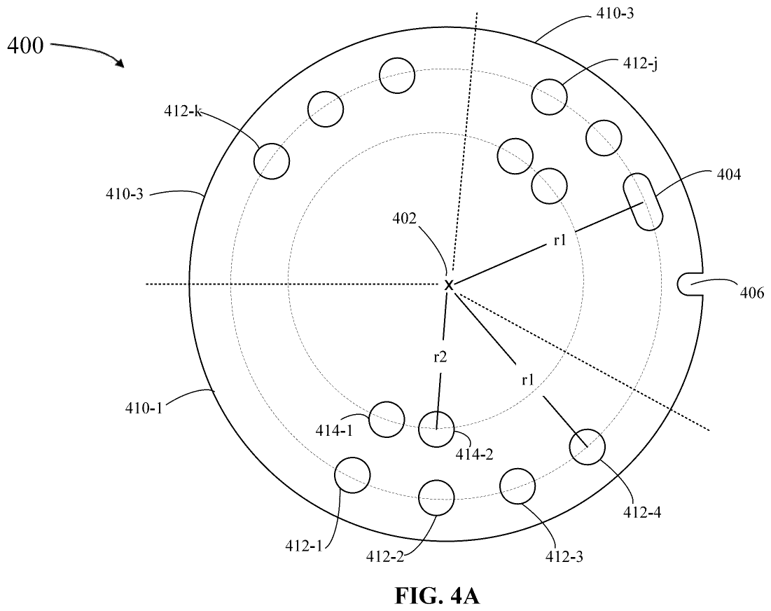

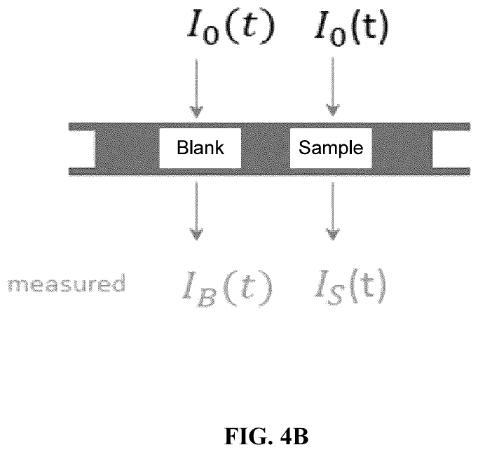

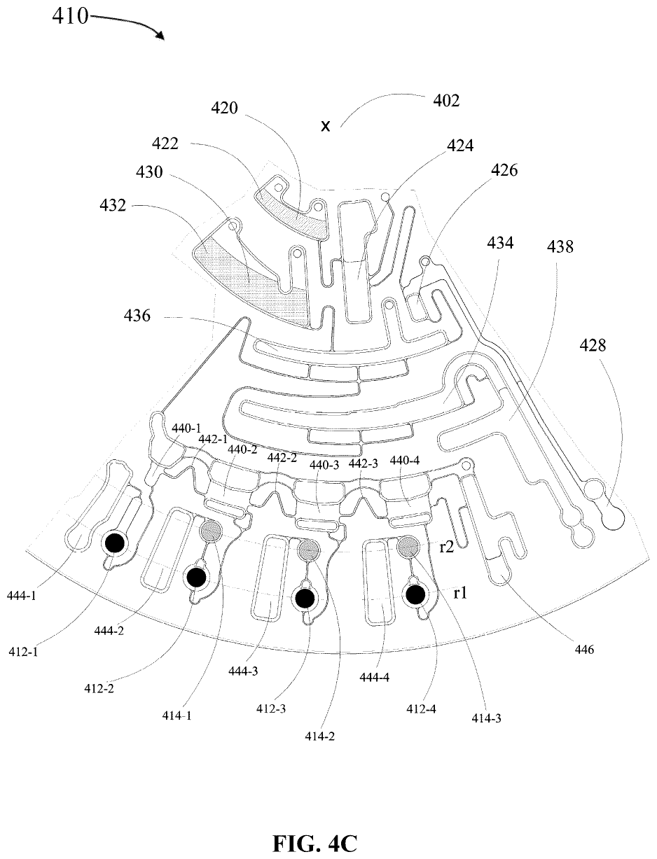

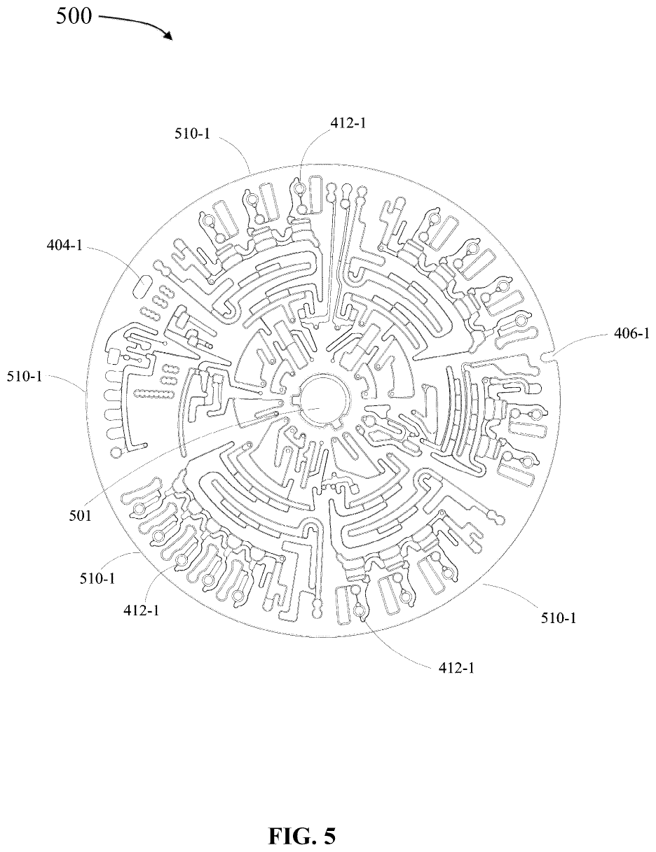

Referring now to the drawings, where like reference numerals indicate like elements throughout, there is shown in FIGS. 1A-1C an exemplary device 100 in accordance with some embodiments of the present disclosure. The device 100 is configured to receive and operate a disc 400 to conduct one or more assays, e.g., determining one or more analytes in a biological sample 420. The device 100 may receive and operate any suitable disc 400 provided that the disc 400 is rotatable around a rotational axis 402 and includes one or more first wells 412 at a first common radius r1 relative to the rotational axis 402 of the disc 400. Examples of such a disc 400 include, but are not limited to, the discs 400 and 500 disclosed herein (which will be described in more detail with respect to FIGS. 4A-4C and FIG. 5) and the discs disclosed in U.S. Provisional Patent Application No. 63/489,422 and U.S. Provisional Patent Application No. 63/489,677, the content of each application is hereby incorporated by reference in its entirety. Each of the one or more first wells (e.g., the first well 412-1 in FIG. 4A) contains one or more reagents disposed therein and is configured to receive an aliquot of a fluid including one or more components of a biological sample 420. The disc 400 may be slid into the device 100 through a slot or opening 102. The slot or opening 102 may be configured to allow sliding the disc 400 in the device 100 during the start of a process and/or retrieving the disc 400 at the end of the process. In some embodiments, the slot or opening 102 is formed on a side of the device 100.

The device 100 is configured to operate the disc 400 to conduct one or more assays, e.g., determining one or more analytes in a biological sample 420. In various embodiments, the device 100 is configured to receive and operate a disc 400 to conduct assays in the category of general chemistry (e.g., homogeneous, liquid-phase assays) by measuring optical absorbance or extinction of one or more components of the biological sample 420. Examples of a biological sample 420 include, but are not limited to, blood, sera, plasma, bone marrow, lymph, saliva, sputum, mucus, respiratory or nasal secretion, oropharyngeal swab, nasopharyngeal swab, oral swab, ductal lavage, bronchoalveolar lavage, cerebrospinal fluid, skin swab, vaginal swab, gastric juice, ascites, peritoneal fluid, pleural fluid, gynecological fluids, pus, perspiration, tears, urine, stool, or any combination thereof. In some embodiments, the biological sample 420 includes blood, such as whole blood. In some embodiments, the biological sample 420 or a component of the biological sample 420 includes plasma. In one embodiment, one or more components of the biological sample 420 comprise K-EDTA anticoagulated whole blood.

The device 100, along with the disc 400, can be used to determine any suitable number of analytes. For instance, in some embodiments, the device 100, along with the disc 400, is configured to determine at least five, at least ten, at least fifteen, at least twenty, at least twenty-five, at least thirty, at least thirty-five, at least forty, at least forty-five, or at least fifty analytes. In some embodiments, the device 100, along with the disc 400, is configured to determine from five to twenty, from ten to thirty, from five to forty, or from twenty to fifty analytes. In some embodiments, the device 100, along with the disc 400, is configured to determine about fifteen, about twenty, about twenty-five, or about thirty analytes. In some embodiments, the device 100, along with the disc 400, is configured to determine a full set of analytes for clinical chemistry. Examples of the one or more analytes to be determined include, but are not limited to, hemoglobin A1C (HbA1C), ketones, aspartate transaminase (AST), alanine transaminase (ALT), lactate, gamma-glutamyltransferase (GGT), glucose, calcium, high-density lipoprotein (HDL), low-density lipoprotein (LDL), sodium ion (Na+), potassium ion (K+), chloride ion (Cl−), total cholesterol (Chol), alkaline phosphatase (ALP), total carbon dioxide (tCO2), total protein, lactate dehydrogenase (LDH), blood urea nitrogen (BUN), direct bilirubin (dBil), total bilirubin (tBil), creatine kinase (CK), creatinine, triglycerides (Trig), albumin, hemolysis, icterus and lipemia (HIL) or any combination thereof.

In some embodiments, the device 100 is configured to enable loading, for instance, by pipette, of a biological sample 420 and reagents on the disc 400. For instance, in some embodiments, the device 100 is configured to rotate the disc 400 to particular positions to allow access to specific loading ports, e.g., by pipette.

In some embodiments, the device 100 is configured to perform the assays on the disc 400 by rotating the disc 400 following a predefined program of speed vs. time. In some such embodiments, controlled centrifugal forces are employed to direct the motion of the biological sample 420 and buffer through channels, wells, chambers or the like on the disc 400, rehydrate lyophilized reagents, mix them, or any combination thereof at the appropriate times. In some embodiments, the device 100 is configured to enable optical measurement in a continuous, semi-continuous or episodic fashion, e.g., allowing optical measurement while the disc 400 is spinning, e.g., at a speed of hundreds of rotations per minute (rpm), thousands rpm, or greater. The optical measurement may be performed at different time windows and/or multiple wavelengths, making it easier to synchronize chemistries and measure the whole time. The optical measurement may be performed at a sampling rate that allows for spatial averaging (e.g., averaging within a reaction well), temporal averaging (e.g., averaging across multiple revolutions).

The device 100 generally includes a motor 110 and an optical measurement unit 120. The motor 110 is configured to rotate the disc 400 received in the device 100 and the optical measurement unit 120 configured to measure optical signals from or through the biological sample 420 on the disc 400. In some embodiments, the disc 400 is coupled with a spindle 14 and the motor 110 rotates the disc 400 through the spindle 14. The motor 110 can be of any suitable type, including but not limited to electric motors. In some embodiments, the motor 110 is a brushless DC electric motor.

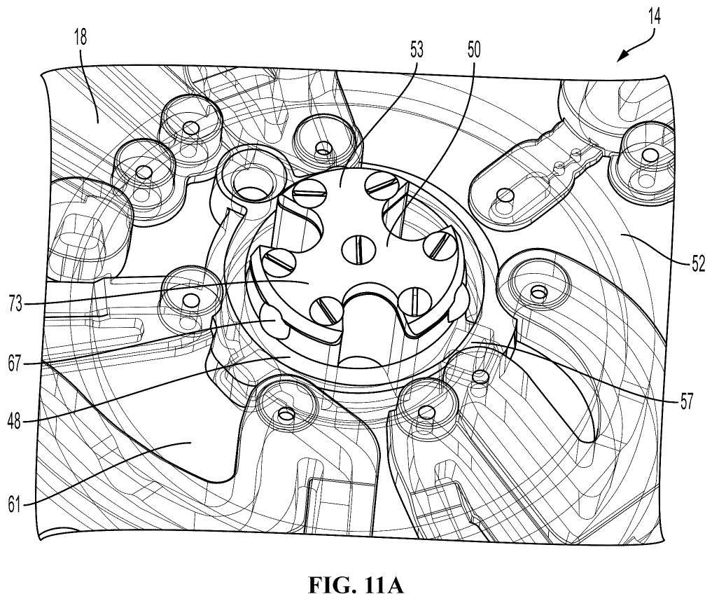

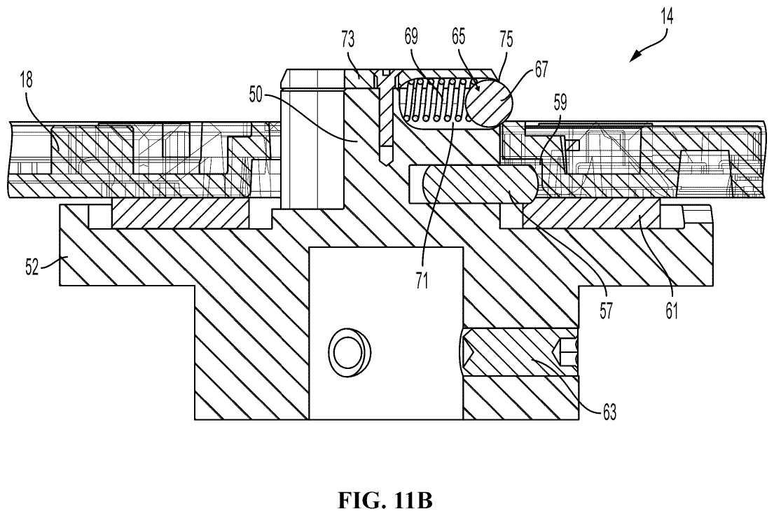

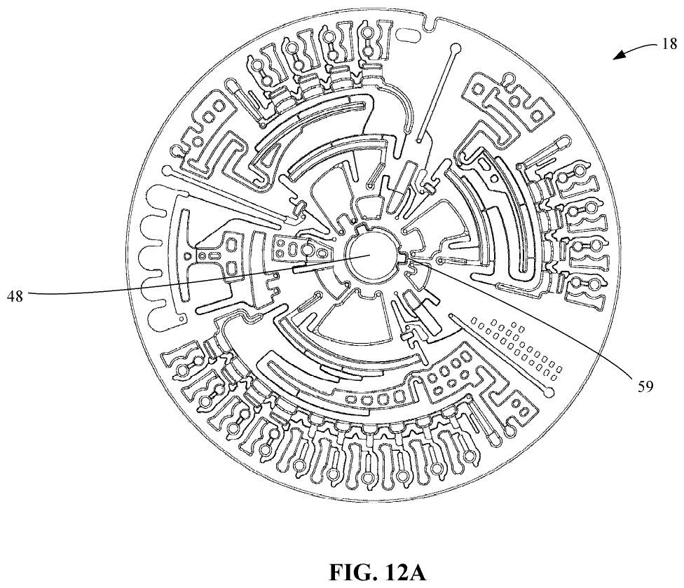

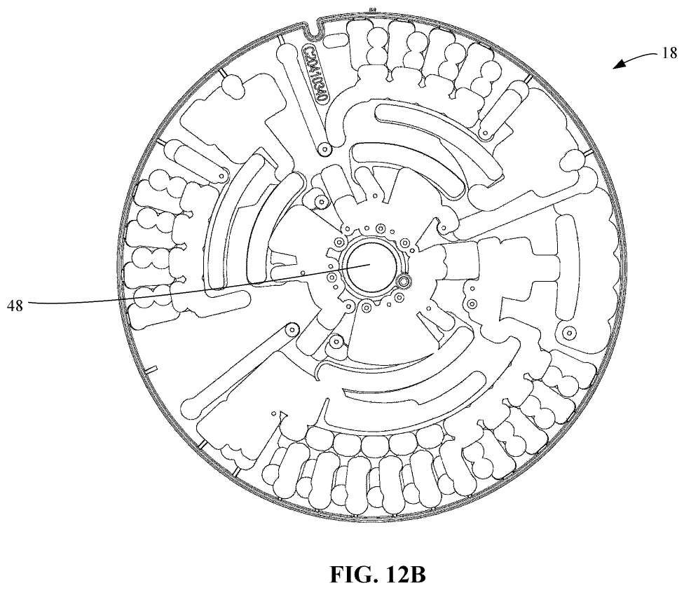

Referring briefly to FIGS. 11A-12B, there are depicted an exemplary spindle 14 in FIGS. 11A and 11B and an exemplary disc 18 in FIGS. 12A and 12B in accordance with some embodiments of the present disclosure. The disc 18 may include an aperture 48, and one or more detents 59. The detent 59 may extend into the disc 18 from the aperture 48. In some embodiments, the detent 59 may protrude from the disc 18. The disc 18 may include other features, such as those disclosed herein with respect to the disc 400 or 500. Similarly, the disc 400 or 500 may include the same or similar features, such as the aperture and detent, disclosed herein with respect to the disc 18.

The spindle 14 may comprise a locking element 50 configured to prevent the disc 18 (or any other disc disclosed herein or the like) from rotating relative to the spindle 14. The locking element 50 may include at least one protrusion 53 extending radially therefrom. The locking element 50 may include at least two protrusions 53 extending radially therefrom. The locking element 50 may include at least four protrusions 53 extending radially therefrom. The locking element 50 may include at least six protrusions 53 extending radially therefrom. The locking element 50 may include at least ten protrusions 53 extending radially therefrom. The spindle 14 may include a collar 52 extending radially therefrom. The collar 52 may be a generally circular shape. The collar 52 may have a diameter greater than that of the aperture 48 to prevent the disc 18 from passing over the collar 52. The collar 52 may include a deformable ring 61 disposed thereon. The deformable ring 61 may be comprised of a rubber or other deformable material. The deformable ring 61 may extend around at least a portion of the collar 52. In some embodiments, the deformable ring 61 extends around only a portion of the collar 52. The deformable ring 61 may provide friction between the spindle 14 and the disc 18 and ensure the transfer of torque from the spindle 14 to the disc 18. The deformable ring 61 may extend from the collar 52 to engage the disc 18 when the disc 18 is disposed on the spindle 14.

The locking element 50 may include at least one locking pin 57 extending therefrom. The locking element 50 may include at least two locking pins 57 extending therefrom. The locking element 50 may include at least five locking pins 57 extending therefrom. The locking element 50 may include at least ten locking pins 57 extending therefrom. The locking pin 57 may extend from the locking element 50 proximate the collar 52. The locking pin 57 may be received in a detent 59 of the disc 18. The bottom of the disc 18 may include at least as many detents 59 as the spindle 14 has locking pins 57. The detent 59 may extend into the disc 18 from the aperture 48. The detent 59 may be shaped and sized to receive at least a portion of the locking pin 57. The locking pin 57 may prevent the disc 18 from moving relative to the spindle 14 during operation of the device 100. In a case where more than one locking pin 57 is included on the spindle 14, the locking pins 57 may be unevenly spaced apart to ensure only one possible orientation of the disc 18 on the spindle 14. This orientation may ensure the disc 18 accurately receives the fluid into sample chambers 422, buffer chambers or other structures.

To ensure the locking pin 57 is aligned with the detent 59 when the disc 18 is received in the device 100, a spindle setting mechanism may be used. The spindle 14 may be coupled to the motor 16 (e.g., the motor 110 in FIG. 1B) with a fastener 63. The spindle 14 may be fixed to the motor 16. The motor 16 may have a starting position, which corresponds to the position of the locking pin 57. The starting position may be electrically programmed into the controller 24 which moves the motor 16 into the starting position before the disc 18 is received in the device 100.

As shown in FIGS. 11A and 11B, the spindle 14 may include a disc lock 65 spaced apart from the collar 52. The disc lock 65 may be spaced apart from the collar 52 such that the disc 18 can fit therebetween. The disc lock 65 may be a ball pin disposed at an end of the locking element 50. The spindle 14 may include as many disc locks 65 as it includes locking pins 57. In some embodiments, there are more disc locks 65 than locking pins 57. In some embodiments, there are less disc locks 65 than locking pins 57. The disc lock 65 may include a ball 67 and a spring 69. The ball 67 and spring 69 may be received in a cavity 71 of the locking element 50. The cavity 71 may be a generally cylindrical space defined by the locking element 50 and a lock top 73 coupled to the end of the locking element 50. The lock top 73 may be fixed to the locking element 50 by, for example, a screw. The spring 69 may urge the ball 67 out of the cavity 71. The cavity 71 may include an opening 75 that has a smaller diameter than the ball 67 to prevent the ball 67 from moving out of the cavity 71. The ball 67 may retract in response to a force by, for example, the disc 18 being disposed on the spindle 14. The ball 67 may extend when the force is released, for example, the disc 18 has been received on the spindle 14. The ball 67 may enact a force on the disc 18 when the disc 18 is received on the spindle 14. The ball 67 may urge the disc 18 toward the collar 52. The lock may prevent the disc 18 from moving relative to the collar 52 along the rotational axis during operation of the device 100.

Referring back to FIGS. 1A-1C, the motor 110 is configured to rotate the disc 400 at controlled speeds or according to a predefined protocol. For instance, the motor 110 may rotate the disc 400 at controlled speeds ranging from about 0 revolutions per minute (rpm) to about 5000 rpm, from about 0 rpm to about 6000 rpm, from about 0 rpm to about 7000 rpm, from about 0 rpm to about 8000 rpm, from about 0 rpm to about 9000 rpm, from about 0 rpm to about 10000 rpm, from about 0 rpm to about 11000 rpm, from about 0 rpm to about 12000 rpm, from about 0 rpm to about 12000 rpm, from about 0 rpm to about 13000 rpm, from about 0 rpm to about 14000 rpm, or from about 0 rpm to about 15000 rpm or higher. In some embodiments, the motor 110 may rotate the disc 400 during a particular period of time (e.g., a detection period) at a speed of at least about 500 rpm, at least about 600 rpm, at least about 700 rpm, at least about 800 rpm, at least about 900 rpm, at least about 1000 rpm, at least about 1200 rpm, at least about 1400 rpm, at least about 1600 rpm, at least about 1800 rpm, at least about 2000 rpm, at least about 2200 rpm, at least about 2400 rpm, at least about 2600 rpm, at least about 2800 rpm, at least about 2900 rpm, at least about 3000 rpm, at least about 3500 rpm, at least about 4000 rpm, at least about 4500 rpm, or at least about 5000 rpm.

In some embodiments, the device 100 or the motor 110 is configured to enable control of rotational speed with a resolution, including but not limited to a resolution of about 50 rpm, about 40 rpm, about 30 rpm, about 25 rpm, about 20 rpm or better. In some embodiments, the device 100 is configured to enable control of rotational speed with an accuracy, including but not limited to an accuracy of about +/−25 rpm, about +/−20 rpm, about +/−15 rpm, about +/−10 rpm or better. In some embodiments, the device 100 is configured to enable control of acceleration and deceleration of the disc 400 rotational movement in a range, including but not limited to a range of about 10 rpm/s to about 4000 rpm/s, about 15 rpm/s to about 4500 rpm/s, about 20 rpm/s to about 5000 rpm/s, about 25 rpm/s to about 5500 rpm/s, about 30 rpm/s to about 6000 rpm/s, about 35 rpm/s to about 6500 rpm/s, or about 40 rpm/s to about 7000 rpm/s.

In some embodiments, the device 100 or the motor 110 is configured to enable control of rotational speed such that a commanded change in speed is achieved within about +/−1 second, about +/−0.9 second, about +/−0.8 second, about +/−0.7 second, about +/−0.6 second, about +/−0.5 second, or less of the nominal time period. In some embodiments where a protocol specifies a ramp up from about 0 rpm to about 600 rpm at 50 rpm/s, the device 100 achieves a speed of about 590 rpm to about 610 rpm within 11.5 to 12.5 seconds.

In various embodiments, the optical measurement unit 120 is configured to enable optical measurement, e.g., measuring optical signals from or through the biological sample 420 on the disc 400. For instance, in some embodiments, the optical measurement unit 120 is configured to measure optical absorbance or extinction through one or more first wells on the disc 400. The measurement can be performed while the disc 400 is stationary or spinning. In some embodiments, the optical measurement unit 120 is configured to enable fast optical acquisition and thus to allow optical acquisition while the disc 400 is spinning at a speed of hundreds rpm, thousands rpm, or greater. Examples of optical acquisition devices and methods include those disclosed in U.S. Patent Application Publication No. 2019/0082961 A1, U.S. Pat. No. 10,761,019 B2, and International Application Publication No. 2019/058308 A1, the content of each application is hereby incorporated by reference in its entirety for all purposes.

In some embodiments, the optical measurement unit 120 is configured to enable signal acquirement in a continuous, semi-continuous or episodic fashion. For instance, in some embodiments, the optical measurement unit 120 is operable to start or restart data acquisition about every 60 seconds, about every 55 seconds, about every 50 seconds, about every 45 seconds, about every 40 seconds, about every 35 seconds, about every 30 seconds, about every 25 seconds, about every 20 seconds, about every 15 seconds, about every 10 seconds, or less. This guarantees adequate temporal resolution for liquid-detection applications, kinetic-assays, diagnostic applications, or any combination thereof.

In some embodiments, during an operation (e.g., a detection period or any other data acquisition period), the optical measurement unit 120 is configured to acquire data at a frequency that guarantees a minimum of readings within one first well for every rotation of the disc 400. The minimum of readings may be determined based on a number of factors, including but not limited to the biological sample 420, the analyte(s) to be determined, the disc 400 (e.g., the size and shape of the first well 412), operation parameters (e.g., the rotation speed of the disc 400), the illumination source(s) 122, the detector(s) 124, or any combination thereof. In some embodiments, the minimum of readings is one, two, three, four, five, six, seven, eight, nine, ten or greater readings within one first well 412 for every rotation of the disc 400. In some embodiments, the minimum of readings is at least three, at least four, at least five, at least six, at least seven, at least eight, at least nine, or at least ten readings within one first well 412 for every rotation of the disc 400. In an embodiment, the minimum of readings is between three and ten readings within one first well for every rotation of the disc 400.

In some embodiments, the optical measurement unit 120 includes one or more illumination sources 122 and one or more detectors 124 arranged in a transmission orientation. That is, the one or more illumination sources 122 and the one or more detectors 124 are disposed optically (not necessarily physically) at opposite sides of the one or more first wells 412 of the disc 400. The one or more illumination sources 122 provides illumination (e.g., light) that impinges on the one or more first wells 412 of the disc 400. The one or more detectors 124 detect the light that has transmitted or passed through the one or more first wells 412. In some embodiments, the one or more illumination sources 122 and the one or more detectors 124 are physically disposed at opposite sides of the one or more first wells 412.

The one or more illumination sources 122 can be any suitable light sources or a combination of different types of light sources. For instance, in some embodiments, the one or more illumination sources 122 include one or more light-emitting diodes (LEDs), one or more superluminescent diodes (SLEDs), one or more narrowband light sources, one or more broadband light sources, one or more lasers, or any combination thereof. In some embodiments, the one or more illumination sources 122 provide illumination at one or more wavelengths from about 280 nm to about 1000 nm. In some embodiments, the one or more illumination sources 122 provide illumination at a plurality of wavelengths. Examples of wavelengths include but are not limited to 340 nm, 405 nm, 456 nm, 505 nm, 545 nm, 580 nm, 600 nm, 610 nm, 645 nm, 660 nm, 720 nm, or any combination thereof.

The control of the illumination beam of illumination sources 122 is important for accurate measurement and detection. An optical axis is defined from each of the illumination sources 122 positioned at the measurement radius, through and perpendicular to the plane of the disc 400, at the measurement radius. In embodiments that include a lens, the lenses may focus and direct the light from the illumination sources 122 to ensure it precisely targets the wells on the disc 400. The lens is positioned centered on the optical axis such that it is between the illumination source 122 and the disc 400. The distance between the lens and the illumination source 122 is fixed to control the intensity and uniformity of the light. This focusing can enhance the intensity and uniformity of the light, improving the sensitivity and accuracy of the detection. An aperture may be centered on the optical axis between the lens and the disc 400 to control the shape and size of the incident light upon the disc 400 at the measurement radius. In embodiments that do not include a lens, diffraction may be leveraged to shape and direct the light beam. In this embodiment, an aperture or series of apertures are centered on the optical axis and positioned between the illumination source 122 and the disc 400 to control the size, shape, and uniformity of the light incident upon the disc 400. The size and shape of the aperture or series of apertures and the distance between them can be fixed to control size, shape, and uniformity of the light incident upon the disc 400. The light beam may compensate for the absence of physical focusing elements.

As used herein, a wavelength generally refers to a wavelength range having a central wavelength and a spectral bandwidth, e.g., a full width at half maximum (FWHM). For instance, a wavelength of 340 nm refers to a wavelength range having a central wavelength of about 340 nm and a spectral FWHM. Similarly, a wavelength of 405 nm refers to a wavelength range having a central wavelength of about 405 nm and a spectral FWHM. The spectral FWHM may be about ±5 nm, about ±10 nm, about ±15 nm, about ±20 nm, about ±25 nm, about ±30 nm, about ±35 nm, about ±40 nm, or more. The spectral FWHM of one wavelength may be the same as or different from the spectral FWHM of another wavelength. An illumination at a wavelength (e.g., with a central wavelength and a spectral FWHM) may be selected to target a particular assay or assays, to reduce the chance of capturing a low-response part of the absorption spectrum of the assay chromophore, to increase the target assay sensitivity, or any combination thereof.

In some embodiments, the one or more illumination sources 122 include a plurality of illumination sources 122, each providing an illumination at a corresponding wavelength in the plurality of wavelengths. In some embodiments, the plurality of illumination sources 122 is disposed circumferentially at a common radius corresponding to the first common radius of the disc 400. This allows for each illumination source 122 to illuminate each of the one or more first wells 412 sequentially while the disc 400 is spinning.

In some embodiments, the one or more detectors 124 includes a plurality of detectors, each configured to detect an optical signal indicative of a corresponding wavelength in the plurality of wavelengths. In some embodiments, the plurality of detectors 124 is disposed circumferentially at a common radius corresponding to the first common radius of the disc 400. In some embodiments, each of the plurality of detectors 124 is optically aligned with a corresponding illumination source in the plurality of illumination sources 122. In some embodiments, each of the plurality of detectors 124 includes an optic element 126 (e.g., lens, series of lenses and/or filter) and a receiver 128. The optical element 126 may be used to focus and direct the beam to ensure it precisely targets the receiver 28. In some embodiments, the receiver 128 includes one or more photodiodes. In some embodiments, the receiver 128 does not include a lens and/or filter.

In some embodiments, one or each detector 124 is configured to receive a beam with a central wavelength and a spectral FWHM targeted for a particular assay or assays. This reduces the chance of capturing a low-response part of the absorption spectrum of the assay chromophore, thereby increasing the effective extinction coefficient and the assay sensitivity. The appropriate spectral bandwidth can be achieved in practice using a combination of illumination sources (e.g., having the one or more illumination sources 122 to provide an illumination with the specific central wavelength and a spectral FWHM as disclosed above) and optical filters (e.g., the optic element 126). For example, in some embodiments, a bandpass filter can be used in conjunction with the one or more illumination sources 122 to more tightly control the central wavelength. Exemplary central wavelengths and methods for some assays are listed in Table I. The spectral FWHMs for these central wavelengths may be about ±5 nm, about ±10 nm, about ±15 nm, about ±20 nm, about ±25 nm, about ±30 nm, about ±35 nm, about ±40 nm, or more.

TABLE I
Optical Readout Parameters

| Assay | Center wavelength (nm) | Assay Method |
|---|---|---|
| Chol | 505 | Endpoint |
| Mg | 505 | Endpoint |
| AST | 340 | Kinetic |
| ALT | 340 | Kinetic |
| Trig | 505 | Endpoint |
| HbA1c | 720 | Endpoint |
| Glucose | 505 | Endpoint |
| Phos | 505 | Endpoint |
| LDH | 340 | Kinetic |
| Lactate | 340 | Endpoint |
| ALP | 405 | Kinetic |
| GGT | 405 | Kinetic |
| CK | 340 | Kinetic |
| TP | 546 | Endpoint |
| Ca | 660 | Endpoint |
| Alb | 610 | Endpoint |
| HDL | 505 | Endpoint |
| BUN | 340 | Kinetic |
| Amylase | 405 | Endpoint |
| Lipase | 580 | Endpoint |
| Ketone | 340 OR 505 | Endpoint |
| CO2 | 340 | Kinetic |
| TBil | 460 | Endpoint |
| Creat | 340 OR 505 | Endpoint |
| Na | 720 & 645 & 505 | Endpoint |
| K | 720 & 645 & 505 | Endpoint |
| Cl | 720 & 645 & 505 | Endpoint |
| HIL: Hemolysis | 546 & 610 | Endpoint |
| HIL-Lipemia | 660 & 720 | Endpoint |
| HIL-Icterus | 460 & 505 | Endpoint |

In some embodiments, at least one detector 124 is configured to receive a beam such that its central wavelength is commensurate with the requirements shown in Table I for one or more assays. In some such embodiments, additionally or optionally, at least one illumination source 122 is configured to provide the illumination with its central wavelength that is commensurate with the requirements shown in Table I for one or more assays. For instance, in some embodiments, a detector 124 is configured to receive a beam having the central wavelength of about 505 nm and spectral FWHM of about 20 nm targeted for Chol, Mg, Trig, HDL, glucose, HDL, Ketone, and/or other assays, and additionally or optionally, an illumination source 122 is configured to provide the illumination with the central wavelength of about 505 nm and spectral FWHM of about 20 nm. In some embodiments, a detector 124 is configured to receive a beam having the central wavelength of about 340 nm and spectral FWHM of about 20 nm targeted for AST, ALT, CK, BUN, CO2, and/or other assays, and additionally or optionally, an illumination source 122 is configured to provide the illumination with the central wavelength of about 340 nm and spectral FWHM of about 20 nm. In some embodiments, each detector 124 is configured to receive a beam such that its central wavelength and spectral FWHM are commensurate with the requirements for a particular assay or assays shown in Table I. In some such embodiments, additionally or optionally, each illumination source 122 is configured to provide an illumination such that its central wavelength and spectral FWHM are commensurate with the requirements for a particular assay or assays shown in Table I. However, the present invention is not limited thereto. Other central wavelengths and spectral FWHM can be used, and other assays can be performed in a similar fashion.

In some embodiments, one or each illumination source 122 provides an illumination where the waist of its beam (e.g., a minimum of the beam) is smaller than a first well 412. In some embodiments, the waist of its beam is smaller than half the dimension of the first well 412. For instance, in embodiments where the first well 412 is circular, the beam waist is smaller than the radius of the first well 412. This minimizes potential contamination of the signal from sides of the first well 412 and thus enhances the sensitivity of measurement. In another embodiment, the waist of its beam is larger than the first well 412. In some embodiments, there is a series of apertures above the illumination source 122 to collimate the beam.

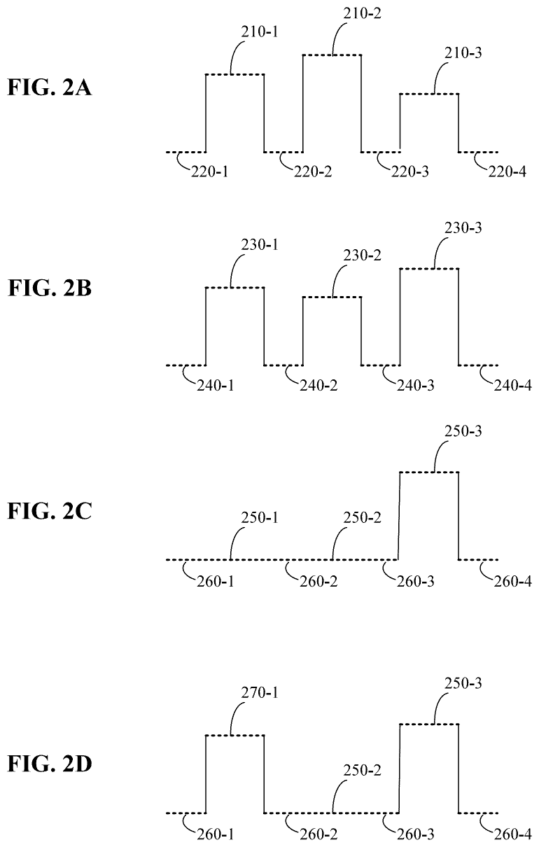

In some embodiments, each illumination source 122 provides an illumination beam that is smaller than each first well 412. In another embodiment, each illumination source 122 provides an illumination beam that is larger than each first well 412. In some such embodiments, each detector 124 produces a plurality of readings per revolution of the disc 400 for each first well 412 during a detection period. For instance, as a non-limiting example, FIGS. 2A, 2B and 2C illustrate readings produced by three detectors 124 for three first wells 412 (e.g., first wells 412-1, 412-2 and 412-3 in FIG. 4A) in one revolution during a detection period. Each detector 124 may be at a common radius relative to the rotational axis of the disc 400. In some embodiments, each detector 124 is at a different radius relative to the rotational axis of the disc 400. In FIG. 2A, the readings 210-1, 210-2, and 210-3 correspond respectively to the optical signals (e.g., absorbance or extinction) from the first wells 412-1, 412-2 and 412-3 detected by a first detector. The readings 220-1, 220-2, 220-3 and 220-4 correspond respectively to the optical signals from the disc 400 space before the first well 412-1, the disc 400 space between the first wells 412-1 and 412-2, the disc 400 space between the first wells 412-2 and 412-3, and the disc 400 space after the first well 412-3 detected by the first detector. In some embodiments, the readings are in the form of voltages (e.g., mV). In some embodiments, the readings are adjusted for gain on the device. The measured voltages are in waveforms because the measurement is performed while the disc 400 is spinning and includes wells-with-sample and spaces/empty wells between wells-with-sample.

Similarly, in FIG. 2B, the readings 230-1, 230-2 and 230-3 correspond respectively to the optical signals from the first wells 412-1, 412-2, 412-3, and 412-4 detected by a second detector 124. The readings 240-1, 240-2, 240-3 and 240-4 correspond respectively to the optical signals from the space before the first well 412-1, the space between the first wells 412-1 and 412-2, the space between the first wells 412-2 and 412-3, and the space after the first well 412-3 detected by the second detector 124. In FIG. 2C, the readings 250-1, 250-2 and 250-3 correspond respectively to the optical signals from the first wells 412-1, 412-2 and 412-3 detected by a third detector 124. The readings 260-1, 260-2, 260-3 and 260-4 correspond respectively to the optical signals from the space before the first well 412-1, the space between the first wells 412-1 and 412-2, the space between the first wells 412-2 and 412-3, and the space after the first well 412-3 detected by the third detector 124.

In some embodiments, for each first well 412, each detector 124 produces at least two readings (e.g., the reading 210-1, etc. includes at least two data points), at least three readings (e.g., the reading 210-1, etc. includes at least three data points), at least four readings, at least five readings, at least six readings, at least seven readings, at least eight readings, at least nine readings, at least ten readings, at least fifteen readings, or at least twenty readings per revolution of the disc 400 during the detection period. In some embodiments, for each first well 412, each detector 124 produces a minimal of at least two readings, at least three readings, at least four readings, at least five readings, at least six readings, at least seven readings, at least eight readings, at least nine readings, or at least ten readings per revolution of the disc 400 during the detection period. In some embodiments, for each first well 412, each detector 124 produces from two to ten readings, from three to twelve readings, or from four to fifteen readings per revolution of the disc 400 during the detection period. This allows for high confidence that the measurements are performed on the first well(s) 412 and for averaging the readings within each first well 412.

A detection period may last for one or more revolutions of the disc 400. For instance, in some embodiments, the detection period lasts for at least two, at least five, at least ten, at least fifteen, at least twenty, at least thirty, at least forty, at least fifty, at least sixty, at least seventy, at least eighty, at least ninety, at least one-hundred, at least two-hundred, at least three-hundred, at least four-hundred, at least five-hundred, at least six-hundred, at least seven-hundred, at least eight-hundred, or at least nine-hundred revolutions of the disc 400. In some exemplary embodiments, the detection period lasts for at least one-thousand, at least two-thousand, at least three-thousand, at least four-thousand, at least five-thousand revolutions, at least six-thousand, at least seven-thousand, at least eight-thousand, or at least nine-thousand revolutions of the disc 400.

In some embodiments, the disc 400 is operated to rotate at a speed such that there is not much time for the chemistry to change in one or more first wells 412. In some such embodiments, the readings produced by the same detector 124 for the same first well 412 may be averaged across multiple revolutions. For instance, in some embodiments, the readings produced by one or each detector 124 for one or each first well 412 may be averaged across two, three, four, five, six, seven, eight, nine, ten or more than ten resolutions. In some embodiments, the readings produced by one or each detector 124 for one or each first well 412 may be averaged across a plurality of revolutions over a predefined time period such as 1, 1.2, 1.4, 1.6, 1.8, or 2 seconds.

The detection period may be at any suitable point of the process. In some embodiments, the detection period corresponds to an endpoint reaction of a corresponding analyte in the one or more analytes of the biological sample 420. Examples of analytes that can be detected at the endpoint reaction include, but are not limited to, hemoglobin A1C (HbA1C), glucose, calcium, high-density lipoprotein (HDL), total cholesterol, total protein, total bilirubin (tBil), creatinine, triglycerides, and albumin. In some embodiments, the detection period corresponds to a kinetic reaction of a corresponding analyte in the one or more analytes of the biological sample 420. Examples of analytes that can be detected during the kinetic reaction include, but are not limited to, aspartate transaminase (AST), alanine transaminase (ALT), gamma-glutamyltransferase (GGT), alkaline phosphatase (ALP), total carbon dioxide (tCO2), or blood urea nitrogen (BUN). In some embodiments, the detection period corresponds to both a kinetic reaction and an end point of a corresponding analyte in the one or more analytes of the biological sample 420.

Referring still to FIGS. 1A-1C, in some embodiments, the device 100 includes (i) a first plate 130, (ii) a first assembly 140, (iii) a second assembly 150, (iv) a third assembly 160, (v) a fourth assembly 170, (vi) an adapter plate 180, or any combination thereof. The first plate 130 is configured for housing the disc 400. As used herein, a plate can be, but do not have to, a thin, smooth, or flat piece. For instance, it may be thick, may be of any suitable shape, may have a compartment to hold or retain other components, or the like. The first plate 130 may include a recess 131 defined therein. The recess 131 may have a generally circular shape and extend around a portion of the first plate 130. The first plate 130 may include a door 132 coupled to an outer edge thereof. The door 132 may extend over the recess 131. The door 132 may pivot relative to the first plate between an open position and a closed position. The disc 400 may be disposed in the recess 131 when the door 132 is in the open position. The disc 400 may be prevented from being removed from the recess 131 when the door 132 is in the closed position. The door 132 may have a length substantially similar to the first plate 130.

The first assembly 140 is disposed at a first side of the first plate 130 (e.g., the lower side in FIG. 1B) and includes the one or more illumination sources 122. In some embodiments, the first assembly 140 includes a first printed circuit board assembly (PCBA) 142. In some embodiments, the one or more illumination sources 122 are mounted on the first PCBA 142.

The second assembly 150 is disposed at a second side of the first plate (e.g., the upper side in FIG. 1B) and includes the one or more detectors 124. In some embodiments, the first assembly 140 includes a second PCBA 152. In some embodiments, the second assembly 150 includes a second plate 154 disposed at the second side of the first plate and configured for mounting the one or more detectors 124. In some embodiments, the second assembly 150 may include a mount 155 fixed on the second plate 154. The mount 155 may be fixed to a side of the second plate 154 opposite the side of the second plate 154 that couples to the first plate 130. The mount 155 may have a generally crescent shape extending around a portion of the second plate 154. In one embodiment, the mount 155 extends fully around the second plate 154. The mount 155 may include one or more apertures 156 extending therethrough. In some embodiments, the mount 155 may include at least two, at least four, at least six, at least eight, at least ten, or more apertures 156. The aperture 156 may extend through the mount 155 and the second plate 154. The aperture 156 may be shaped and sized to receive at least a portion of the optic element 126 (e.g., lens and/or filter). In some embodiments, the aperture 156 may receive the entire optic element 126.

The third assembly 160 is disposed at or adjacent to the first plate 130 and configured for regulating a temperature of the biological sample 420 or the fluid including the one or more components of the biological sample 420 in the disc 400. For instance, in some embodiments, the third assembly 160 is a PCBA including a radiation heat source 162. The third assembly 160 is disposed below the disc 400 and configured to allow the disc 400 to rotate relative to the third assembly 160. Rotation of the disc 400 relative to the third assembly 160 serves as the air circulation mechanism, creating air movement and circulation between the disc 400 and the radiation heat source 162. When the radiation heat source 162 is on, the air movement and circulation average any difference in heating across the third assembly 160 and the disc 400. When the radiation heat source 162 is off, the air movement and circulation average any difference in cooling across the third assembly 160 and the disc 400. This results in very uniform heating or cooling and keeping the temperature of the disc 400 in a tight window around a desired temperature. Because the air movement and circulation are generated by rotation of the disc 400, there is no need for any air circulation. This reduces the complexity of the device 100 and the manufacturing cost of the device 100.

In some embodiments, the desired temperature may be a targeted temperature range between about 34° C. and about 40° C., between about 35° C. and about 39° C., between about 36° C. and about 38° C., or about 37° C. In some embodiments, the desired temperature may be a particular range of 37° C.±1° C. or 37° C.±0.5° C. In some embodiments, the desired temperature may be a particular range of 36° C.±1° C. or 36° C.±0.5° C. In some embodiments, the third assembly 160 or the radiation heat source 162 is configured such that it takes no longer than about 60 seconds, about 50 seconds, about 40 seconds, about 30 seconds or less from the time of introduction of the disc 400 into the device 100 to reach the target temperature range. In some embodiments, the third assembly 160 or the radiation heat source 162 is configured such that it takes no longer than about 60 seconds, about 50 seconds, about 40 seconds, about 30 seconds or less from the time of introduction of liquids (e.g., buffer or sample) on the disc 400 to the time when the liquids reach the operating temperature. The disc 400 and/or liquid may be refrigerated before the time of introduction.

The fourth assembly 170 is disposed at or adjacent to the first plate 130 and configured for determining a location of the disc 400 relative to a home location. In some embodiments, the home location may correspond to the one or more illumination sources 122, the one or more detectors 124, the motor 110, or any combination thereof. In some embodiments, the fourth assembly 170 includes a PCBA 172 and a sensor mounted or embedded in the PCBA 172. In some embodiments, a single PCBA 172 houses the one or more illumination sources 122 and the one or more detectors 124. In some embodiments, the fourth assembly 170 is configured to detect a location of a positioning structure of the disc 400 (e.g., the positioning structure 406 of the disc 400 in FIG. 4A). In some embodiments, the location of the positioning structure is referred herein as the zero position on the disc 400. This allows for resetting the disc 400 and the disc 400 angular position. In some embodiments, the fourth assembly 170 is configured for performing this location process with a repeatability of about ≤0.25, about ≤0.20, about ≤0.15, or about ≤0.1 degree from run to run on a standardized disc 400.

In some embodiments, the device 100 is configured to enable positioning of the disc 400 such that any point on the disc 400 is within about +/−200 μm, about +/−180 μm, about +/−160 μm, about +/−140 μm, about +/−120 μm, about +/−100 μm, or less of its nominal position. This ensures that sample 420 or reagents can be introduced effectively into the disc 400 and that the disc 400 is in proper alignment.

The adapter plate 180 is configured for mounting the device 100 to an instrument or a mounting bracket. The device 100 may be run independently or simultaneously with any other devices in the instrument.

In some embodiments, the first plate 130, the second plate 154, and/or other components of the device 100 are configured to hold the disc 400 in position during the operation. In some embodiments, the first plate 130, the second plate 154, and/or other components of the device 100 are configured to apply a downward force on the disc 400 such that the resultant frictional forces are sufficient to enable the disc 400 to rotate without slippage. In some embodiments, the first plate 130, the second plate 154, and/or other components of the device 100 are configured to allow loading of liquid on the disc 400 without causing any permanent change (e.g., titling) in the position of the disc 400.

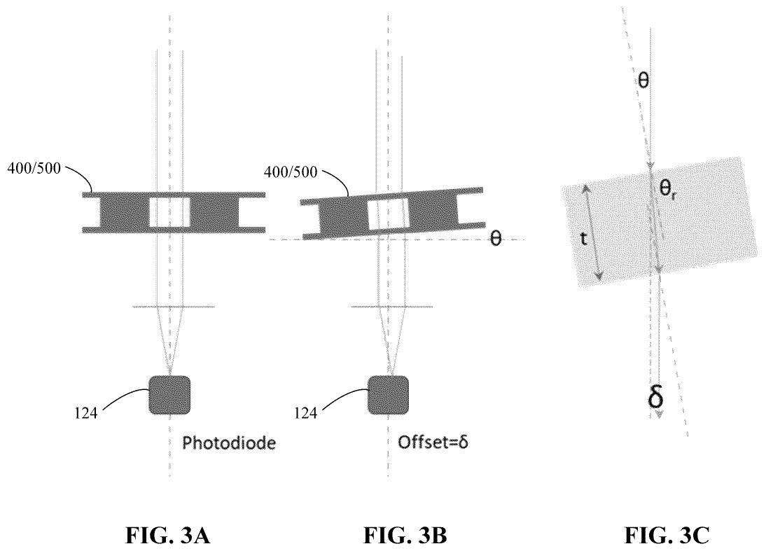

In some embodiments, the device 100 is configured to maintain vibrations at a low-level during rotation of the disc 400 to reduce or eliminate adverse effects on sample 420, e.g., maintaining vibration at or below a level such that they do not lead to red blood cell (RBC) lysis. In some embodiments, the device 100 is configured to maintain vibrations at a low-level during rotation of the disc 400 such that the effective tilt of the disc 400 is no more than a certain degrees (e.g., 2.5 degrees). This ensures that the tilt does not significantly affect the optical measurements. For instance, by way of example, FIG. 3A illustrates photon flux (e.g., the illumination) impinged on a disc 400 (e.g., the disc 400 or 500 disclosed herein) with no offset. FIG. 3B illustrates the photon flux impinged on the disc 400 with an offset due to the tilt of the disc 400. FIG. 3C illustrates a model that considers a structure of plastic-liquid-plastic as a single slab. In FIGS. 3B and 3C, θ denotes the tilt of the structure and δ denotes the offset of the photon flux on the photodiode (e.g., the detector 124 of the device 100). The offset of the photon flux δ can be calculated as follows:

$${θ}_{r} = {sin}^{- 1} ( \frac{{n}_{a}}{{n}_{p}} sin θ )$$

$$δ = t \frac{sin ( θ - {θ}_{r} )}{cos {θ}_{r}}$$

Assuming that the plastic has a refractive index of 1.49 and the liquid has a refractive index of 1.33, this leads to approximately 5 m per degree tilt per mm slab thickness. As such, 2.5-degree tilt in a 4 mm thick slab corresponds to 50 μm shift in the center beam hitting the detector 124. Thus, the tilt of ≤2.5 degrees in a 4 mm thick slab will not significantly affect the photon flux on the detector 124, even in the presence of an aperture.

The device 100 can include other additional, optional, or alternative components. For instance, in some embodiments, the device 100 may include one or more components for detecting or determining mechanical failures. The detection or determination may be implemented using feedback from a rotary encoder. The mechanical failures include but are not limited to (i) if the device fails to successfully home the disc 400, i.e., reset the position of the disc 400, (ii) if the device fails to successfully reach the commanded angular position for the disc 400, (iii) if the device fails to successfully reach the target rotational speed within the specified time, (iv) if the disc 400 slips during rotational motion, or any combination thereof.

In some embodiments, the device 100 may include one or more components for detecting or determining if the temperature of the disc 400 is out of the specified range.

In some embodiments, the device 100 may include one or more components for detecting or determining presence or absence of liquid in different structures on the disc 400. The detection or determination may be implemented using a camera-system capable of imaging the disc 400 or based on optical measurement (e.g., absorbance measurement). The detection and determination of the presence or absence of liquid in different structures on the disc 400 may include, but are not limited to, detecting or determining (i) if there are sufficient volumes of sample 420 and reagents, which may be conducted by imaging the stationary disc 400 prior to spinning to validate accuracy of loaded sample 420 and buffer volumes, (ii) if there is incomplete injection of sample 420 (e.g., plasma) into a dilution structure, which may be conducted by imaging the stationary disc 400 at the end of the assay to detect residual liquid, if any, in the plasma metering chamber, (iii) if there is incorrect metering of buffer, which may be conducted by absorbance measurement in a buffer overflow chamber 438 to verify buffer metering during a plasma-priming-and-metering step, (iv) if there are structural defects in shelf stabilized reagents (e.g., lyophilized reagents in a form of beads) such as melted or disintegrated reagents due to humidity exposure or mechanical shock, which may be detected by imaging prior to spinning, (v) if there is incomplete rehydration of the reagents such as clumps or aggregates of particulate matter, which may be detected by imaging at the end of the assay, (vi) or any combination thereof.

The device 100 can be used to receive and operate any suitable disc 400 having any suitable number of first wells 412 to conduct one or more assays, e.g., determining one or more analytes in a biological sample 420. For instance, a disc 400 may have one, two, three, four, five, more than five, more than ten, more than fifteen, more than twenty, more than twenty-five, more than thirty, more than thirty-five, more than forty, more than forty-five, or more than fifty first wells 412. A disc 400 having multiple first wells 412 is preferable for running reactions and/or detection of multiple analytes in parallel.

### Exemplary Discs

Referring to FIG. 4A, there is shown an exemplary disc 400 in accordance with some embodiments of the present disclosure. The disc 400 is rotatable around a rotational axis 402 and includes a plurality of first wells, such as first wells 412-1, 412-2, 412-3, 412-j, and 412-k. In some embodiments, the disc 400 includes at least five first wells, at least ten first wells, at least fifteen first wells, at least twenty first wells, at least twenty-five first wells, at least thirty first wells, at least forty first wells, or at least fifty first wells 412. A first well 412 can be of any suitable shape, including but not limited to circle, oval, or polygonal, and different first wells may or may not have the same shape or size. As a non-limiting example, FIG. 4A illustrates the first wells 412 with substantially the same circular shape and size.

Each first well 412 is at a first common radius, designated by “r1” in the figure, relative to the rotational axis 402 of the disc 400. In some embodiments, the first common radius r1 may be greater than about 35 mm, greater than about 40 mm, greater than about 45 mm, greater than 50 mm, greater than 55 mm, or greater than 60 mm. In some embodiments, the first common radius r1 may be between about 25 mm and about 45 mm, between about 30 mm and about 50 mm, between about 35 mm and about 55 mm, or between about 40 mm and about 60 mm. In some embodiments, the first common radius r1 may be between about 43 mm and about 50 mm.

A disc 400 may be divided into a plurality of slices, such as two, three, four, five or more than five slices. A slice is a portion of the disc 400. It can be of any suitable shape or size and can be located in any suitable place in the disc 400. For instance, a slice can be but does not have to be of a fan shape. Different slices can be identical or different in terms of their shapes, sizes, and/or structures in the slices. For instance, a slice may be larger than another slice, may have different number of structures, and/or may have structures with different functions. Moreover, a slice may or may not have a first well 412. For instance, in some embodiments, each slice has at least one first well 412. In some embodiments, at least one slice has no first well 412. As a non-limiting example, FIG. 4A illustrates that the disc 400 is divided into the slices 410-1, 410-2 and 410-3 and that each of the slices 410-1, 410-2 and 410-3 includes at least one first well, e.g., the slice 410-1 including the first well 412-1, the slice 410-2 including the first well 412-j, and the slice 410-3 including the first well 412-k.

A first well 412 is configured to receive an aliquot of a fluid including one or more components of the biological sample 420. A first well 412 may or may not contain the same amount of the one or more components of the biological sample 420 as another first well 412. In some embodiments, at least some of the first wells 412 contain the same amount of the one or more components of the biological sample 420. In some embodiments, at least some of the first wells 412 contain different amounts of the one or more components of the biological sample 420. For instance, in some embodiments, the first wells 412-1 and 412-2 contain the same amount of the one or more components of the biological sample 420. In some embodiments, the first wells 412-1 and 412-j contain different amounts of the one or more components of the biological sample 420.

In some embodiments, the fluid is a mixture including a dilution buffer, e.g., the fluid is a mixture of which the biological sample 420 or the component(s) of the biological sample 420 is diluted with a buffer. The fluid may include the dilution buffer at a dilution factor of about 1:1 to about 1:200. For instance, in some embodiments, the fluid includes the dilution buffer at a dilution factor of about 1:10 to about 1:50, about 1:20 to about 1:60, about 1:30 to about 1:70, about 1:40 to about 1:80, about 1:50 to about 1:100, about 1:60 to about 1:120, about 1:80 to about 1:120, about 1:100 to about 1:150, about 1:120 to about 1:180.

A first well 412 may or may not receive a fluid having the same dilution buffer or in the same dilution factor as another first well 412. In some embodiments, at least some first wells 412 receive the fluid including the same dilution buffer. In some embodiments, at least some first wells 412 receive the fluid including the same dilution buffer at a different factor. In some embodiments, at least some of first wells 412 receive the fluid including different buffers (e.g., different types of buffers). For instance, in some embodiments, the first wells 412-1 and 412-2 receive the fluid having the same dilution buffer and at the same dilution factor. In some embodiments, the first wells 412-1 and 412-j receive the fluid having different dilution buffers. In some embodiments, the first wells 412-j and 412-k receive the fluid having the dilution buffer but at different dilution factors.

A first well 412 is configured to receive or contain one or more reagents disposed therein. For instance, in some embodiments, a first well 412 is configured to contain one or more reagents disposed therein during manufacturing of the disc 400. The one or more reagents may be lyophilized and may be in bead form. In some embodiments, the disc 400 or at least a portion of the disc 400 is made of an injection molded thermoplastic piece with one or more cavities as the first well, where the one or more reagents (e.g., lyophilized beads) are placed.

A first well 412 may or may not receive or contain the same reagent(s) as another first well 412. In some embodiments, at least some of the first wells 412 contain a same reagent, at least some of the first wells 412 contain different reagents, or both. For instance, in some embodiments, the first wells 412-1 and 412-j contain a same reagent. In some embodiments, the first wells 412-1 and 412-2 contain different reagents.

A disc 400 may include at least one second well 414 positioned radially inward of the one or more first wells 412. The number of second wells 414 may or may not be the same as the number of the first wells 412, and the shape and size of a second well 414 may or may not be the same as a first well 412. In some embodiments, a disc 400 may have the same number of first and second wells, i.e., each first well 412 having a corresponding second well 414 and vice versa. In some embodiments, a disc 400 may have fewer second wells 414 than first wells 412, i.e., at least one first well 412 does not have a corresponding second well 414. The first and second wells are referred herein as reaction wells.

By way of example, FIG. 4A illustrates the disc 400 having a plurality of second wells 414, e.g., second wells 414-1 and 414-2. In some embodiments, each second well 414 is positioned at a second common radius, designated by “r2” in the figure, relative to the rotational axis 402 of the disc 400. The second common radius r2 is shorter than the first common radius r1 of the first wells 412. In some embodiments, the second common radius may be smaller than about 30 mm, smaller than about 35 mm, smaller than about 40 mm, smaller than about 45 mm, or smaller than 50 mm.

Each second well 414 is configured to receive or contain one or more reagents disposed therein. For instance, in some embodiments, each second well 414 is configured to contain one or more reagents disposed therein during manufacturing of the disc 400. The one or more reagents may be lyophilized and may be in bead form. A second well 414 may or may not receive or contain the same reagent(s) as another second well 414.

The first and second wells allow for performing single and multiple reaction assays in the same disc 400. For instance, in some embodiments, the disc 400 has at least one structure with a single well (e.g., the first well 412-1) for a single-reaction assay and at least one structure with two wells (e.g., the first well 412-2 and second well 414-1, or the first well 412-3 and second well 414-2) for a two-reaction assay on the same disc 400. These may minimize the reaction volume to be very close to the minimum volume required to reconstitute a lyophilized bead (e.g., the structure for a single-reaction assay uses ~½ volume of the structure for a two-reaction assay). In addition, this provides extreme flexibility when designing new discs. However, it should be noted that a single-reaction assay can be performed in the first well 412-2, 412-3, or 412-4.

In some embodiments, the disc 400 includes a positioning structure 406 for positioning the disc 400 or resetting the disc 400 relative to the device 100. The positioning structure 406 can be of any suitable shape and size and can be located at any suitable position in the disc 400. As a non-limiting example, FIG. 4A illustrates that the positioning structure 406 is a notch at the edge of the disc 400.

In some embodiments, the disc 400 includes one or more wells other than the first/second wells. For instance, in some embodiments, the disc 400 includes a reference well 404 at the first common radius r1 relative to the rotational axis of the disc 400. The reference well 404 may or may not be of the same shape or size as a first or second well. In some embodiments, the reference well 404 is blank or filled with a standard test solution or other standard material(s) with known properties (e.g., known absorbance) to serve as a reference.

For instance, during a detection period, one or more optical signals from the reference well 404 can be detected, using the one or more detectors 124, to produce at least one reading for the reference well per revolution of the disc 400. The at least one reading for the reference well 404 can then be used as a reference to register the reading(s) for each first well 412 based on the geometric information of each first well 412 relative to the reference well 404. This makes data processing simple and robust. For instance, it does not require any actual finding of the reading(s) (e.g., peaks) for any first well in the algorithm. It will not miss the reading(s) for any first well 412, and it will eliminate any false findings for any first well 412. It guarantees that the reading(s) registered for each first well 412 is indeed the reading(s) for that particular first well 412.

The reading(s) for the reference well 404 can also be used to determine the accuracy and precision of the optical components (e.g., the detector 124) of the device 100. For instance, as a non-limiting example, FIG. 4B illustrates illumination and optical measurement on a first well 412 and the reference well 404. In the figure, I₀ denotes the intensity of light (e.g., the illumination from an illumination source 122) incident on the first well 412 and the reference well 404. I_{B} denotes the amount of light transmitted through the reference well 404 and detected by a detector 124. I_{S} denotes the amount of light transmitted through the first well 412 and detected by the same detector 124. The absorbance A_{B} through the reference well 404, the absorbance A_{S} through the first well 412, and the absorbance A_{blanked} by the air or standard test solution or any material in the reference well 404 can be calculated as follows:

$${A}_{B} = - log \frac{{I}_{B}}{{I}_{0}}$$

$${A}_{S} = - log \frac{{I}_{S}}{{I}_{0}}$$

$${A}_{blanked} = {A}_{S} - {A}_{B} = - log \frac{{I}_{S}}{{I}_{B}}$$

As can be seen, the blanked absorbance A_{blanked} is independent of the intensity of incident light I₀. Since the reference well 404 is blank or filled with a standard test solution or material with known absorbance, A_{blanked} can be used to determine the accuracy and precision of the optical components such as the detector(s) 124 of the device 100.

The disc 400 can include other structures. For instance, as a non-limiting example, FIG. 4C illustrates a slice 410, e.g., one of the slices 410-1, 410-2 and 410-3 of the disc 400, in accordance with some embodiments of the present disclosure. In some embodiments, the slice 410 includes a sample chamber 422, a separation chamber 424 and a sample metering chamber 426. The sample chamber 422 is positioned radially inward of the one or more first wells 412 and configured for loading a biological sample 420 420. The separation chamber 424 is in fluidic communication with the sample chamber 422 and configured for separating the biological sample 420 into multiple components. For instance, in some embodiments, the biological sample 420 is blood and one of the multiple components is plasma. The sample metering chamber 426 is in fluidic communication with the separation chamber 424 and configured for metering one or more components of the biological sample 420. For instance, in embodiments where the biological sample 420 is blood and a component of the biological sample 420 is plasma, the separation chamber 424 is configured to metering plasma. In some embodiments, the slice 410 also includes a sample overflow chamber 428 to receive any excess amount of plasma or other component(s) of the biological sample 420 or to allow any excess amount of plasma or other component(s) of the biological sample 420 to flow through.

In some embodiments, the slice 410 includes a buffer chamber 432, a buffer metering chamber 434 and a mixing chamber 436. The buffer chamber 432 is positioned radially inward of the one or more first wells 412 and configured for loading a buffer 430. The buffer metering chamber 434 is in fluidic communication with the buffer chamber 432 and configured for metering the buffer. The mixing chamber 436 is in fluidic communication with the sample metering chamber 426 and the buffer metering chamber 434 and configured for mixing the metered one or more components of the biological sample 420 with the metered buffer. The mixing produces a fluid including the one or more components of the biological sample 420 and the buffer at a dilution factor. In some embodiments, the slice 410 also includes a buffer overflow chamber 438 to receive any excess amount of the buffer or to allow to flow through.

In embodiments where a disc 400 includes multiple slices, the type or amount of the buffer loaded to one slice may or may not be the same as the buffer loaded to another slice, and the fluid produced in one slice may or may not have the dilution factor as the fluid produced in another slice. For instance, the type or amount of the buffer loaded to the slice 410-1 may or may not be the same as the buffer loaded to the slice 410-1 of the disc 400, and the fluid produced in the slice 410-1 may or may not have the dilution factor as the fluid produced in the slice 410-1 of the disc 400.

In some embodiments, a fluid produced in a slice may include the dilution buffer at a dilution factor of about 1:1 to about 1:200. For instance, in some embodiments, the fluid includes the dilution buffer at a dilution factor of about 1:10 to about 1:50, about 1:20 to about 1:60, about 1:30 to about 1:70, about 1:40 to about 1:80, about 1:50 to about 1:100, about 1:60 to about 1:120, about 1:80 to about 1:120, about 1:100 to about 1:150, about 1:120 to about 1:180.

In some embodiments, a slice may include one or more aliquoting chambers 440 in fluidic communication with the mixing chamber 436 and configured for aliquoting the fluid into one or more aliquots, where each of the one or more first wells 412 is in fluidic communication with a corresponding aliquoting chamber 440 to receive an aliquot of the fluid from the corresponding chamber 440. For instance, as a non-limiting example, FIG. 4C illustrates the slice 410 including multiple aliquoting chambers, e.g., aliquoting chambers 440-1, 440-2, 440-3 and 440-4. The aliquoting chambers 440-1, 440-2, 440-3 and 440-4 are positioned radially inward of the first wells 412-1, 412-2, 412-3 and 412-4. The aliquoting chambers 440-1, 440-2, 440-3 and 440-4 are in fluidic communication with the mixing chamber 436 and configured for aliquoting the fluid into multiple aliquots, one aliquot for each of the first wells 412-1, 412-2, 412-3 and 412-4. In some embodiments, each first well 412 is in fluidic communication with a corresponding aliquoting chamber 440, e.g., the first well 412-1 is in fluidic communication with the aliquoting chamber 440-1, to receive an aliquot of the fluid from the corresponding chamber 440.

In some embodiments, adjacent aliquoting chambers 440 are connected with each other by a siphon structure. For instance, adjacent aliquoting chambers 440-1, 440-2 are connected with each other by the siphon structure 442-1, adjacent aliquoting chambers 440-2, 440-3 are connected with each other by the siphon structure 442-2, and adjacent aliquoting chambers 440-3, 440-4 are connected with each other by the siphon structure 442-3.

In some embodiments, a slice includes one or more pneumatic chambers 44 in fluidic communication with the one or more first wells 412. For instance, as a non-limiting example, FIG. 4C illustrates the slice 410 including multiple pneumatic chambers, e.g., pneumatic chambers 444-1, 444-2, 444-3 and 444-4. The pneumatic chamber 444-1 is connected to the first well 412-1 through the second well 414-1. The pneumatic chamber 444-2 is connected to the first well 412-2 through the second well 414-2. The pneumatic chambers 444-3 and 444-4 are respectively connected to the first wells 412-3 and 412-4. In some embodiments, the slice 410 includes a diluted sample overflow chamber 446 to receive any excess amount of the fluid, i.e., the one or more components of the biological sample 420 diluted with the buffer, or to allow any excess amount of fluid to flow through.

FIG. 5 illustrates an exemplary disc 500 in accordance with some embodiments of the present disclosure. Disc 500 is similar to disc 400 except that disc 500 may include a slice (e.g., a portion) that does not include any first well. The disc 500 includes a plurality of slices, such as slices 510-1, 510-2, 510-3 and 510-4. Each of the slices 510-1, 510-2 and 510-3 includes one or more first wells 412. The slice 510-4, however, does not include any first well 412. Instead, the slice 510-4 includes other structures configured for achieving other functions, such as for assays in the category of electrolytes based on fluorescence measurement while the disc 500 is stationary.

### Exemplary Processes

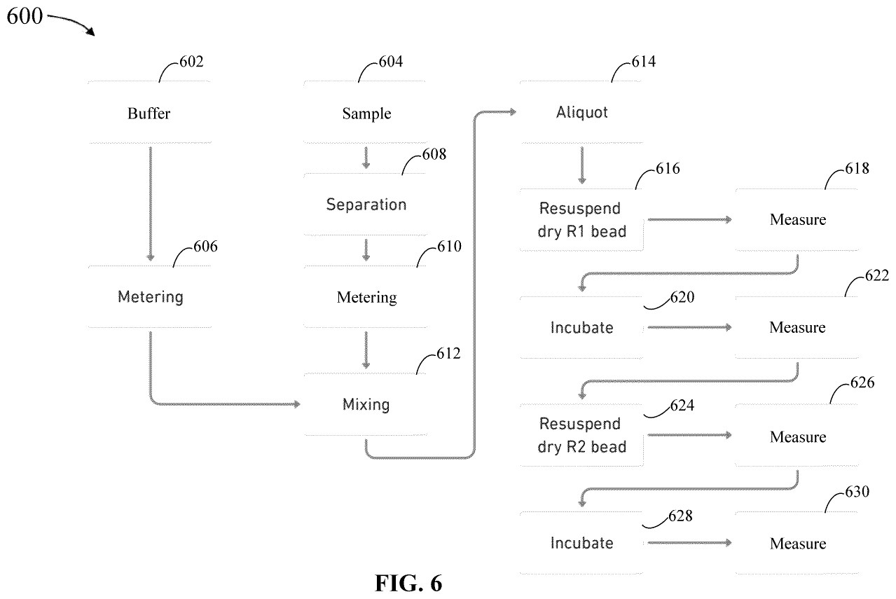

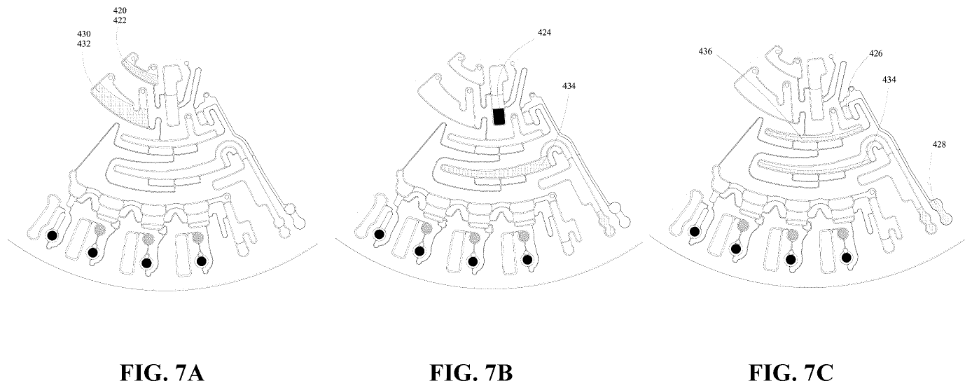

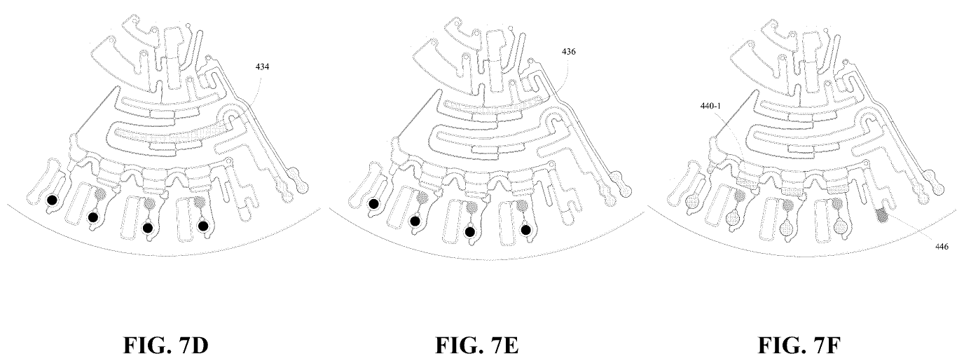

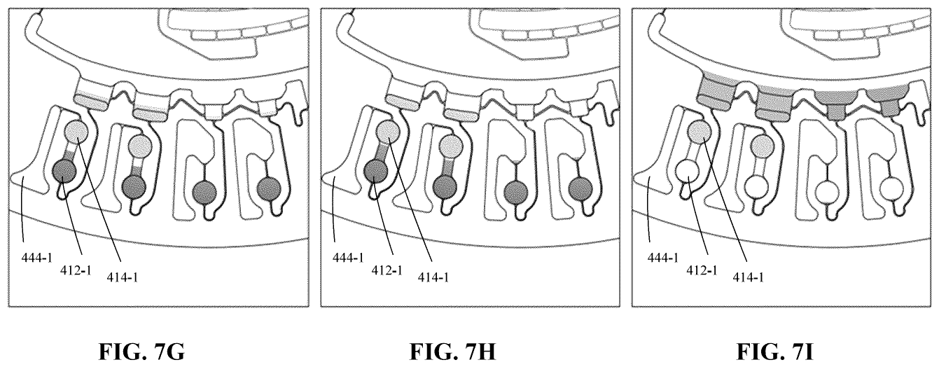

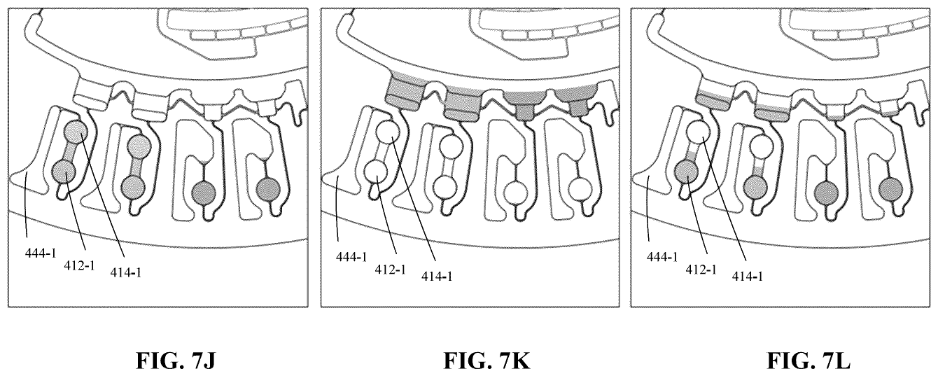

Referring to FIG. 6 and FIGS. 7A-7L, there is shown an exemplary process 600 for determining one or more analytes in a biological sample 420 in accordance with some embodiments of the present disclosure. It should be noted that the processes disclosed herein and exemplified in the process 600 can be, but do not have to be, executed in full or in the order as they are presented.

Referring to blocks 602 and 604, in some embodiments, a buffer and a biological sample 420 are loaded to a disc 400. For instance, as a non-limiting example, FIG. 7A illustrates that the buffer 430 (e.g., water) is loaded to the buffer chamber 432 and the biological sample 420 420 (e.g., blood) is loaded to the sample chamber 422. Loading of the buffer and biological sample 420 can be conducted simultaneously or sequentially, and can be conducted, for instance, by pipette.

Referring to blocks 606 and 608, in some embodiments, the disc 400 is rotated according to a predefined profile to separate the biological sample 420, to meter the buffer, or both. As a non-limiting example, FIG. 7B illustrates that by rotating the disc 400, the biological sample 420 flows from the sample chamber 422 to the separation chamber 424 and separates into multiple components. In some embodiments, the biological sample 420 is whole blood that separates into plasma (radially inward) and cellular fractions (radially outward). The buffer flows from the buffer chamber 432 to the buffer metering chamber 434. In some embodiments, the buffer flows from the buffer chamber 432, through the mixing chamber 436, into the buffer metering chamber 434. In some embodiments, excess buffer, if any, flows into the buffer overflow chamber 438.

Referring to block 610, in some embodiments, the disc 400 is rotated according to a predefined profile to meter one or more components (e.g., plasma) of the biological sample 420. In some embodiments, the rotational speed of the disc 400 is decreased for metering the component(s) of the biological sample 420. As a non-limiting example, FIG. 7C illustrates that as the rotational speed of the disc 400 is decreased, the component(s) of the biological sample 420 in the separation chamber 424 flows to the sample metering chamber 426. Once the sample metering chamber 426 is completely filled, any excess component(s) of the biological sample 420 flows to the sample overflow chamber 428. In some embodiments, the decrease of the rotational speed, along with other structures in the disc 400, also causes the metered buffer flows from the buffer metering chamber 434 to the mixing chamber 436.

Referring to block 612, in some embodiments, the disc 400 is rotated according to a predefined profile to dilute the metered one or more components (e.g., plasma) of the biological sample 420 with the metered buffer. In some embodiments, the rotational speed of the disc 400 is alternately decreased and increased for diluting and/or mixing the metered component(s) of the biological sample 420 with the metered buffer. For instance, as a non-limiting example, FIGS. 7D and 7E illustrate that as the rotational speed of the disc 400 is increased, the metered component(s) flows from the sample metering chamber 426 to the mixing chamber 436. Then as the rotational speed of the disc 400 is decreased, the metered buffer flows from the buffer metering chamber to the mixing chamber 436. Increasing and decreasing the rotational speed of the disc 400 will then cause the mixture of the metered buffer and metered component(s) of the biological sample 420 to flow back and forth between the buffer metering chamber 434 and mixing chamber 436. Increasing and decreasing of the rotational speed of the disc 400 can be repeated until the solution is thoroughly mixed. This produces a diluted sample mixture, i.e., the fluid with the component(s) of the biological sample 420 diluted with the buffer.

Referring to block 614, in some embodiments, the disc 400 is rotated according to a predefined profile to aliquot the diluted sample mixture into one or more aliquots and to transfer one aliquot of the diluted sample mixture to each first well 412. In some embodiments, the rotational speed of the disc 400 is initially decreased and then increased. In embodiments where the disc 400 includes a second well such as the second well 414, the disc 400 is rotated in this step within a speed range such that the diluted sample mixture does not reach the second well. For instance, as a non-limiting example, FIG. 7F illustrates that the rotational speed of the disc 400 is initially decreased to prime the connecting channel between the mixing chamber 436 and aliquoting chambers 440. The rotational speed of the disc 400 is then increased to transfer one aliquot of the diluted sample mixture into each of the first wells 412-1, 412-2, 412-3 and 412-4. In this step, the mixture does not reach the second well 414-1 or 414-2 due to the back pressure in the pneumatic chamber 444-1 or 444-2. In some embodiments, excess mixture, if any, flows to the diluted sample overflow chamber 446.

Referring to block 616, in some embodiments, the diluted sample mixture transferred to each first well 412 dissolves and/or suspends the reagents disclosed therein. This process is illustrated in FIG. 7G.

Referring to blocks 618, 620 and 622, in some embodiments, the disc 400 is rotated according to a predefined profile to mix the diluted sample mixture with the reagents (e.g., dissolved lyophilized reagents) and to perform optical measurement when desired. In some embodiments, the rotational speed of the disc 400 is increased and decreased to cause the diluted sample mixture flowing back and forth between the first wells 412 and aliquoting chambers 440. For instance, as a non-limiting example, FIGS. 7H and 7I illustrate that the increase and decrease of the rotational speed of the disc 400 causes the trapped air in the pneumatic chambers 444 to expand and contract, forcing the diluted sample mixture flowing back and forth between the first wells 412 and aliquoting chambers 440. The increase and decrease of the rotational speed of the disc 400 can be repeated until adequate mixing of the diluted sample mixture with the reagents is achieved. This produces a first reaction mixture. When adequate mixing is achieved or at any time points or periods when desired, the first reaction mixture is transferred back to the first wells 412 for optical measurement. The optical measurement is performed while the disc 400 is rotating.

Referring to block 624, in some embodiments where the disc 400 includes at least one second well (e.g., the second well 414), the disc 400 is rotated according to a predefined profile to transfer the mixture (e.g., the diluted sample mixture if no reaction occurs in the first well 412 or the first reaction mixture if some reactions have occurred in the first well 412) to the second well 414. In some embodiments, this is achieved by increasing the rotational speed of the disc 400. For instance, as a non-limiting example, FIG. 7J illustrated that the increase of the rotational speed of the disc 400 causes the trapped air in the pneumatic chambers 444 to contract, forcing the mixture into the second wells 414-1 and 414-2 and/or dissolving the reagents disclosed therein.

Referring to blocks 626, 628 and 630, in some embodiments, the disc 400 is rotated according to a predefined profile to mix the mixture with the reagent(s) disclosed in the second well(s) and to perform optical measurement when desired. In some embodiments, the rotational speed of the disc 400 is increased and decreased to cause the mixture flowing back and forth between the first wells 412, the second well(s) 414 and aliquoting chambers 440. For instance, as a non-limiting example, FIGS. 7K and 7L illustrate that the increase and decrease of the rotational speed of the disc 400 causes the trapped air in the pneumatic chambers 444 to expand and contract. The expansion and contraction of the trapped air in the pneumatic chamber 444-1 or 444-2 forces the mixture flowing back and forth between the second well 414-1 or 414-2, the first well 412-1 or 412-2, and the aliquoting chamber 440-1 or 440-2. This will produce a second reaction mixture. The expansion and contraction of the trapped air in the pneumatic chamber 444-3 or 444-4 forces the mixture flowing back and forth between the first well 412-3 or 412-4, and the aliquoting chamber 440-3 or 440-4. The increase and decrease of the rotational speed of the disc 400 can be repeated until adequate mixing of the mixture with the reagent(s) disposed in the second well is achieved. When adequate mixing is achieved or at any time points or periods when desired, the second reaction mixture is transferred back to the first wells 412-1 and 412-2, and the first reaction mixture is transferred back to the first wells 412-3 and 412-4 for optical measurement. The optical measurement is performed while the disc 400 is rotating.

In some embodiments, the rotation of the disc 400 is stopped once the optical measurement is complete and/or the disc 400 is discarded.

### Exemplary Methods

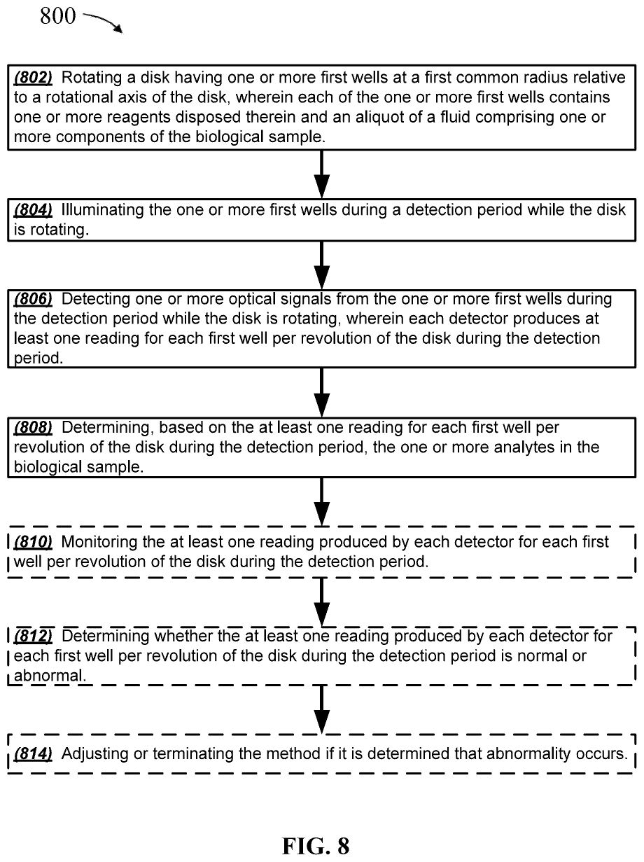

Referring to FIG. 8, there is shown a flowchart illustrating an exemplary method 800 for determining one or more analytes in a biological sample 420 in accordance with some embodiments of the present disclosure. In the flowchart, the preferred parts of the method are shown in solid line boxes, whereas additional, optional, or alternative parts of the method are shown in dashed line boxes. It should be noted that the processes disclosed herein and exemplified in the flowchart can be, but do not have to be, executed in full or in the order as they are presented.

Referring to block 802, in some embodiments, the method 800 includes rotating a disc 400 and the discs disclosed in U.S. Provisional Patent Application No. 63/489,422 and U.S. Provisional Patent Application No. 63/489,677, the content of each application is hereby incorporated by reference in its entirety. In some embodiments, the disc 400 is similar to the disc 18 or 500 disclosed herein. The disc 400 includes one or more first wells (e.g., the first well 412) at a first common radius (e.g., r1) relative to a rotational axis 402 of the disc 400. Each of the one or more first wells 412 contains one or more reagents disposed therein and an aliquot of a fluid including one or more components of a biological sample 420. The biological sample 420 may be any suitable sample such as those disclosed herein. In some embodiments, the biological sample 420 includes blood and one of the components is plasma. In some embodiments, the disc 400 includes a plurality of first wells 412 spaced apart circumferentially from each other.

In some embodiments, the disc 400 includes at least one second well (e.g., the second well 414) positioned at a second common radius (e.g., r2) relative to the rotational axis 402 of the disc 400. In some embodiments, the second common radius r2 is shorter than the first common radius r1. In some embodiments, the disc 400 includes a reference well (e.g., the reference well 404) at the first common radius r1 relative to the rotational axis 402 of the disc 400.

In some embodiments, the disc 400 is placed in a device, such as the device 100 disclosed herein, and rotated by the motor 110 of the device 100. In some embodiments, the disc 400 is rotated during a detection period at a speed of at least about 500 revolutions per minute (rpm), at least about 600 rpm, at least about 700 rpm, at least about 800 rpm, at least about 900 rpm, at least about 1000 rpm, at least about 1200 rpm, at least about 1400 rpm, at least about 1600 rpm, at least about 1800 rpm, at least about 2000 rpm, at least about 2200 rpm, at least about 2400 rpm, at least about 2600 rpm, at least about 2800 rpm, at least about 2900 rpm, at least about 3000 rpm, at least about 3500 rpm, at least about 4000 rpm, at least about 4500 rpm, or at least about 5000 rpm.

Referring to block 804, in some embodiments, the method 800 includes illuminating the one or more first wells 412 during the detection period while the disc 400 is rotating, for instance, using the one or more illumination sources 122 of the device 100. In some embodiments, the illumination beam of each of the one or more illumination sources 122 is smaller than each of the one or more first wells 412. In some embodiments, the one or more illumination sources 122 provide illumination at a plurality of wavelengths, such as those listed in Table I. Each of the plurality of wavelengths corresponds to one or more respective analytes.

Referring to block 806, in some embodiments, the method 800 includes detecting one or more optical signals from the one or more first wells 412 during the detection period while the disc 400 is rotating, for instance, using the one or more detectors 124 of the device 100. Accordingly, each detector produces at least one reading for each first well 412 per revolution of the disc 400 during the detection period. In embodiments where the illumination beam is smaller than each of the one or more first wells 412, each of the one or more detectors may produce a plurality of readings for each of the one or more first wells per revolution of the disc 400 during the detection period. The plurality of readings for each of the one or more first wells 412 per revolution of the disc 400 during the detection period may include at least two readings, at least three readings, at least four readings, at least five readings, at least six readings, at least seven readings, at least eight readings, at least nine readings, at least ten readings, at least fifteen readings, or at least twenty readings.

In some embodiments, for each of the one or more first wells 412 per revolution of the disc 400 during the detection period, the detecting of the one or more optical signals detects light transmitted through each of the one or more first wells, thereby producing the at least one reading that is indicative of light absorption by each of the one or more first wells.

In some embodiments where the disc 400 includes a plurality of first wells 412 spaced apart circumferentially from each other, the detecting of the one or more optical signals also detects one or more optical signals from each spacing between adjacent first wells 412 in the plurality of first wells 412 during the detection period while the motor 110 is rotating the disc 400. Accordingly, each detector 124 produces at least one reading for each spacing between adjacent first wells 412 in the plurality of first wells 412 per revolution of the disc 400 during the detection period.

In some embodiments, where the disc 400 includes a reference well 404, the method 800 includes detecting one or more optical signals from the reference well during the detection period while the disc 400 is rotating the disc 400, using the same detector(s) that detects the one or more optical signals from the one or more first wells 412. Accordingly, each detector 124 produces at least one reading for the reference well 404 per revolution of the disc 400 during the detection period. In some embodiments, the at least one reading for the reference well 404 is used as a reference, for instance, for registering the reading(s) produced for the first well(s) 412, for checking the accuracy and precision of the optical components such as the detector 124, or the like.

In some embodiments, the method 800 uses multiple detectors 124, each detecting an optical signal indicative of the one or more analytes at a particular wavelength, such as those listed in Table I.

Referring to block 808, in some embodiments, the method 800 includes determining, based on the at least one reading for each first well 412 per revolution of the disc 400 during the detection period, the one or more analytes in the biological sample 420. In some embodiments, the determining of the one or more analytes in the biological sample 420 is based on Beer-Lambert's law.

In some embodiments wherein each detector 124 produces a plurality of readings for each first well 412 per revolution of the disc 400 during the detection period, the determining of the one or more analytes in the biological sample 420 may include averaging the plurality of readings for each first well 412 per revolution of the disc 400 during the detection period.

In some embodiments, the detection period lasts for a plurality of revolutions. In some such embodiments, the determining of the one or more analytes in the biological sample 420 may include averaging the at least one reading for each of the one or more first wells 412 per revolution of the disc 400 during the detection period across at least a subset of the plurality of revolutions. For instance, in some embodiments, the determining of the one or more analytes in the biological sample 420 may average the at least one reading across two to ten revolutions, three to twelve revolutions, or four to fifteen revolutions. In some embodiments, the determining of the one or more analytes in the biological sample 420 may average the at least one reading across three, four, five, six, seven, or eight revolutions.

In some embodiments, for each of the one or more first wells 412, the determination of the one or more analytes in the biological sample 420 may include calculating one or more optical densities based on the at least one reading for the reference well 404 and the at least one reading for each first well 412 produced by each of the one or more detectors 124. For instance, in some embodiments, the readings (e.g., I_{S} in FIG. 4A) for wells-with-sample (e.g., any first well 412 containing the biological sample 420) and readings (e.g., I_{S} in FIG. 4A) for empty wells which serve as reference values (e.g., the reference well 404 or any first well 412 that does not contain sample) are identified, for instance, by registering the reading(s) for each first well 412 based on the geometric information of each first well 412 relative to the reference well 404. Reference value determination is not limited thereto and may be determined from a variety of sources including via an air read, diluted biological sample 420 wells, known instrument specific values or other sources. In some embodiments, the readings for the same well are averaged per revolution or across multiple revolutions, e.g., the spatial averaging and temporal averaging disclosed herein are performed on the readings for the same well if applicable. The optical density (OD) is then calculated as OD=log 10 (I_{S}/I_{B}) for every well. In some embodiments, the determination of the one or more analytes may involve directly converting the raw readings to analyte concentration without estimating the optical density.

In some embodiments, for an endpoint assay, the OD value is compared against a pre-established calibration curve (e.g., a curve with known concentration vs. known OD curve). In some embodiments, the pre-established calibration curve is in the form of y=mx+b, where y denotes the optical density and x denotes the concentration.

In some embodiments, for a kinetic assay, the measurement is performed over time to generate a graph of an OD (dependent variable) vs. time (independent variable). The slope of this graph is then calculated and compared against a pre-established calibration curve (e.g., a curve with known concentrations vs. known slope of OD curves). In some embodiments, this pre-established calibration curve is in the form of a=mx+n, where a denotes the slope and x denotes the concentration.

Referring to blocks 810-814, in some embodiments, the method 800 includes, additionally or optionally, monitoring the at least one reading produced by each detector 124 for each first well 412 per revolution of the disc 400 during the detection period. In some embodiments, the method 800 includes, additionally or optionally, determining whether the at least one reading produced by each detector 124 for each first well 412 per revolution of the disc 400 during the detection period is normal or abnormal. In some embodiments, the method 800 includes, additionally or optionally, adjusting or terminating the method if it is determined that abnormality occurs. The abnormality may be due to leakage, cross contamination, incorrect or inaccurate reagent (e.g., wrong reagent placed in the wrong well during the manufacturing of the disc 400), or any combination thereof.

For instance, suppose that the correct readings for three wells (e.g., the first wells 412-1, 412-2 and 412-3 in FIG. 4A) should be the readings 250-1, 250-2 and 250-3 as illustrated in FIG. 2C. However, the readings measured and produced (e.g., by a detector 124 of the device 100) for the three wells are the readings 270-1, 250-2 and 250-3 illustrated in FIG. 2D. The reading 270-1 is different from the correct reading 250-1. This may indicate the occurrence of abnormality due to leakage, cross contamination, incorrect or inaccurate reagent, or any other malfunctions.

Accordingly, in some embodiments, the method is performed to read every well on every wavelength and revolution. This allows for high quality control to ensure that every reagent (e.g., lyophilized bead) is placed in the right position and that there is no leakage and no contamination during the process.

The method 800 may include other additional, optional or alternative steps. For instance, in some embodiments, prior to a first detection period, the method 800 includes rotating the disc 400 according to a first predefined profile to promote mixing of the aliquot of the fluid with the one or more reagents disposed in each of the one or more first wells. In some embodiments, the first predefined profile includes alternately accelerating and decelerating rotation of the disc 400 within a first speed range. In some embodiments where the disc 400 includes at least one second well positioned at a second common radius different than the first common radius of the one or more first wells 412, the method 800 may include rotating the disc 400 according to a second predefined profile to promote mixing of the aliquot of the fluid with the one or more reagents disposed in each of the at least one second well 414. The rotating of the disc 400 according to the second predefined profile may be performed subsequent to the first detection period and prior to a second detection period that detects one or more optical signals. In some embodiments, the second predefined profile includes alternately accelerating and decelerating rotation of the disc 400 within a second speed range.

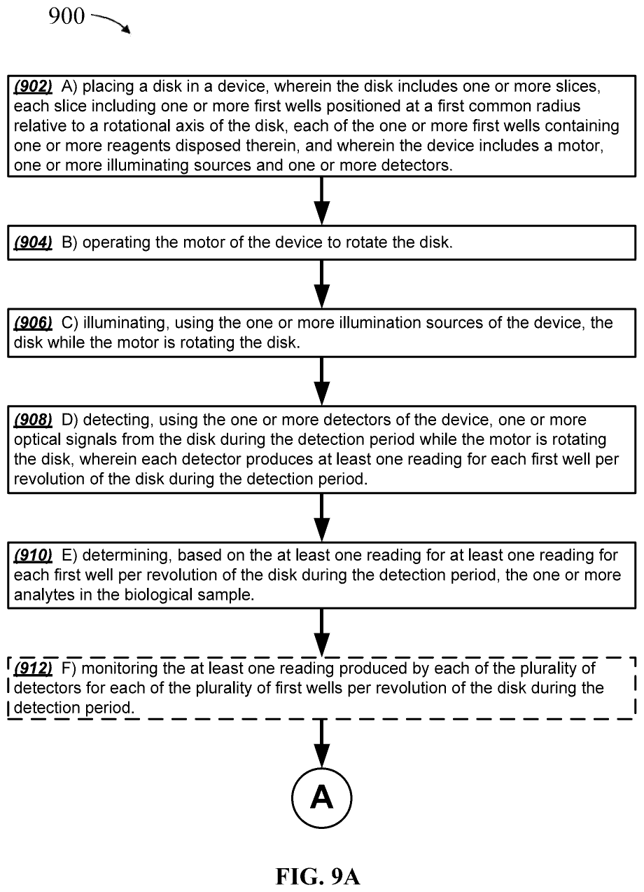

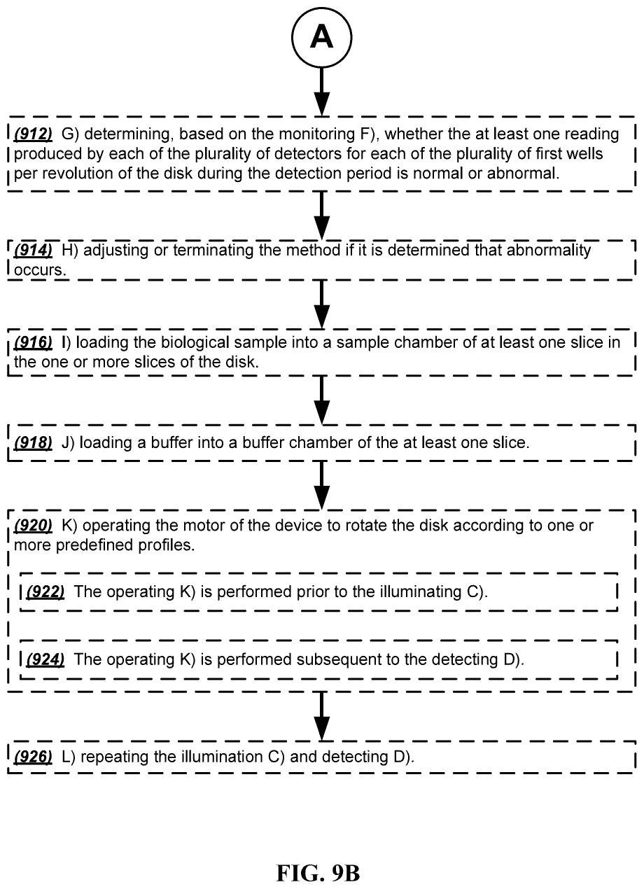

Referring to FIGS. 9A and 9B, there is shown a flowchart illustrating an exemplary method 900 for determining one or more analytes in a biological sample 420 in accordance with some embodiments of the present disclosure. In the flowchart, the preferred parts of the method are shown in solid line boxes, whereas additional, optional, or alternative parts of the method are shown in dashed line boxes. It should be noted that the processes disclosed herein and exemplified in the flowchart can be, but do not have to be, executed in full or in the order as they are presented.

Referring to block 902, in some embodiments, the method 900 includes A) placing a disc 400 in a device, wherein the disc 400 includes one or more slices, each slice 410 including one or more first wells 412 positioned at a first common radius r1 relative to a rotational axis 402 of the disc 400, each of the one or more first wells 412 containing one or more reagents disposed therein, and wherein the device includes a motor 110, one or more illuminating sources 122 and one or more detectors 124. In some embodiments, the disc 400 includes a reference well 404 at the first common radius r1 relative to the rotational axis 402 of the disc 400. In some embodiments, the one or more first wells 412 of the at least one slice 410 include a plurality of first wells 412 spaced apart circumferentially from each other.

In some embodiments, the disc 400 is one of the disc 400 and 500 disclosed herein and the discs disclosed in U.S. Provisional Patent Application No. 63/489,422 and U.S. Provisional Patent Application No. 63/489,677, the content of each application is hereby incorporated by reference in its entirety. In some embodiments, the disc 400 is similar to the disc 400 or 500 disclosed herein. In some embodiments, the device is the device 100 disclosed here or similar to the device 100. The biological sample 420 may be any suitable sample such as those disclosed herein. In some embodiments, the biological sample 420 includes blood and one of the components is plasma.

Referring to block 904, in some embodiments, the method 900 includes B) operating the motor 110 of the device to rotate the disc 400. The operating B) may rotate the disc 400 at any suitable speed range and may depend on the type of the biological sample 420, the disc 400, the analytes to be analyzed, and/or other parameters. For instance, in some embodiments, the operating B) rotates the disc 400 during the detection period at a speed of at least about 500 revolutions per minute (rpm), at least about 600 rpm, at least about 700 rpm, at least about 800 rpm, at least about 900 rpm, at least about 1000 rpm, at least about 1200 rpm, at least about 1400 rpm, at least about 1600 rpm, at least about 1800 rpm, at least about 2000 rpm, at least about 2200 rpm, at least about 2400 rpm, at least about 2600 rpm, at least about 2800 rpm, at least about 2900 rpm, at least about 3000 rpm, at least about 3500 rpm, at least about 4000 rpm, at least about 4500 rpm, or at least about 5000 rpm.

Referring to block 906, in some embodiments, the method 900 includes C) illuminating, using the one or more illumination sources of the device, the disc 400 while the motor 110 is rotating the disc 400. In some embodiments, the one or more illumination sources 122 are configured such that an illumination beam of each of the one or more illumination sources 122 is smaller than each of the one or more first wells 412 of the at least one slice. In some embodiments, the one or more illumination sources 122 provide illumination at a plurality of wavelengths (e.g., the wavelengths disclosed herein), wherein each of the plurality of wavelengths corresponds to one or more respective analytes (e.g., the analytes disclosed herein).

Referring to block 908, in some embodiments, the method 900 includes D) detecting, using the one or more detectors 124 of the device, one or more optical signals from the disc 400 during the detection period while the motor 110 is rotating the disc 400, wherein each detector 124 produces at least one reading for each first well 412 per revolution of the disc 400 during the detection period.

In some embodiments where an illumination beam of each of the one or more illumination sources 122 is smaller than each of the one or more first wells 412 of the at least one slice, each of the one or more detectors 124 produces a plurality of readings for each of the one or more first wells 412 of the at least one slice per revolution of the disc 400 during the detection period. In some embodiments, the plurality of readings for each of the one or more first wells 412 per revolution of the disc 400 during the detection period includes at least two readings, at least three readings, at least four readings, at least five readings, at least six readings, at least seven readings, at least eight readings, at least nine readings, at least ten readings, at least fifteen readings, or at least twenty readings.

In some embodiments where the disc 400 includes a reference well 404 at the first common radius r1 relative to the rotational axis 402 of the disc 400, the detecting D) includes detecting, using the one or more detectors 124, one or more optical signals from the reference well 404 during the detection period while the motor 110 is rotating the disc 400, wherein each of the one or more detectors 124 produces at least one reading for the reference well 404 per revolution of the disc 400 during the detection period, and wherein the at least one reading for the reference well 404 is used as a reference. For instance, the at least one reading for the reference well 404 per revolution of the disc 400 during the detection period may be used for registering the reading(s) produced for the first well(s) 412, for checking the accuracy and precision of the optical components such as the detector 124, or the like.

In some embodiments where the one or more first wells 412 of the at least one slice include a plurality of first wells 412 spaced apart circumferentially from each other, the detecting D) includes detecting, using the one or more detectors 124, one or more optical signals from each spacing between adjacent first wells in the plurality of first wells 412 during the detection period while the motor 110 is rotating the disc 400, wherein each of the one or more detectors 124 produces at least one reading for each spacing between adjacent first wells in the plurality of first wells 412 per revolution of the disc 400 during the detection period.

In some embodiments, the one or more detectors 124 includes a plurality of detectors 124, each configured to detect an optical signal indicative of the one or more respective analytes (e.g., the analytes disclosed herein) at the corresponding wavelength (e.g., the wavelength or wavelengths disclosed herein).

Referring to block 910, in some embodiments, the method 900 includes E) determining, based on the at least one reading for at least one reading for each first well 412 per revolution of the disc 400 during the detection period, the one or more analytes in the biological sample 420. In some embodiments where each of the one or more detectors 124 produces a plurality of readings for each of the one or more first wells 412 of the at least one slice per revolution of the disc 400 during the detection period, the determining E) includes averaging the plurality of readings for each of the one or more first wells 412 of the at least one slice per revolution of the disc 400 during the detection period. In some embodiments, the detection period lasts for a plurality of revolutions. In some such embodiments, the determining E) includes averaging the at least one reading for each of the one or more first wells 412 of the at least one slice per revolution of the disc 400 during the detection period across at least a subset of the plurality of revolutions. In some embodiments, the subset of the plurality of revolutions has from two to ten revolutions, from three to twelve revolutions, or from four to fifteen revolutions. In some embodiments, the subset of the plurality of revolutions has three, four, five, six, seven, or eight revolutions. In some embodiments, the determining E) includes calculating one or more optical densities based on the at least one reading for the reference well 404 and the at least one reading for each first well 412 produced by each of the one or more detectors 124.

Referring to blocks 912-914, in some embodiments, the method 900 includes F) monitoring the at least one reading produced by each of the plurality of detectors 124 for each of the plurality of first wells 412 per revolution of the disc 400 during the detection period. In some embodiments, the method 900 includes F) determining, based on the monitoring F), whether the at least one reading produced by each of the plurality of detectors 124 for each of the plurality of first wells 412 per revolution of the disc 400 during the detection period is normal or abnormal. In some embodiments, the method 900 includes H) adjusting or terminating the method if it is determined that abnormality occurs.

Referring to block 916, in some embodiments, the method 900 includes I) loading the biological sample 420 into a sample chamber 422 of at least one slice in the one or more slices of the disc 400. In some embodiments, the loading I) loads the biological sample 420 into the sample chamber 422 of each of the one or more slices of the disc 400. The loading I) may be performed before the placing A) or subsequent to the placing A). In some embodiments, the loading I) is performed subsequent to the placing A).

Referring to block 918, in some embodiments, the method 900 includes J) loading a buffer into a buffer chamber 432 of the at least one slice. In some embodiments, the loading J) loads the buffer into the buffer chamber 432 of each of the one or more slices of the disc 400. The loading J) may be performed before the placing A) or subsequent to the placing A). In some embodiments, the loading J) is performed subsequent to the placing A).

Referring to blocks 920-924, in some embodiments, the method 900 includes K) operating the motor 110 of the device 100 to rotate the disc 400 according to one or more predefined profiles. The operating K) according to a predefined profile may be performed at any suitable time period. For instance, the operating K) according to a predefined profile may be performed prior to the illuminating C) or subsequent to the detecting D). In some embodiments, the operating K) includes operating one or more predefined profiles prior to the illuminating C), operating one or more predefined profiles subsequent to the detecting D), or any combination thereof.

In some embodiments, the motor 110 of the device 100 is operated, prior to the illuminating C), to rotate the disc 400 according to one or more predefined profiles to: 1) transfer the biological sample 420 from the sample chamber 422 to a separation chamber 424 of the at least one slice, 2) separate the biological sample 420 into multiple components, 3) transfer the buffer from the buffer chamber 432 to a buffer metering chamber 434, 4) meter the buffer, 5) transfer one or more components of the biological sample 420 to a sample metering chamber 426 of the at least one slice, 6) metering the one or more components of the biological sample 420, 7) transfer the metered buffer from the buffer metering chamber 434 to a mixing chamber 436 of the at least one slice, 8) transfer the metered one or more components of the biological sample 420 to the mixing chamber 436 of the at least one slice, 9) promote mixing of the metered one or more components of the biological sample 420 with the metered buffer, thereby producing the fluid including the one or more components of the biological sample 420 and buffer at a defined dilution factor, 10) transfer the fluid to one or more aliquoting chambers 440 of the at least one slice, 11) divide the fluid into one or more aliquots, 12) transfer an aliquot of the fluid to each of the one or more first wells 412 of the at least one slice, 13) promote mixing of the aliquot of the fluid with the one or more reagents disposed in each of the one or more first wells 412, or any combination thereof. The promotion of the mixing may be achieved by operating the motor 110 of the device 100 to alternately increase or decrease the rotational speed of the disc 400 (e.g., alternately accelerating and decelerating rotation of the disc 400) within a speed range.

In some embodiments, the motor 110 of the device 100 is operated, prior to the illuminating C), to rotate the disc 400 according to a first predefined profile to: (i) transfer the biological sample 420 from the sample chamber 422 to a separation chamber 424 of the at least one slice, (ii) separate the biological sample 420 into multiple components, (iii) transfer the buffer from the buffer chamber 432 to a buffer metering chamber 434, (iv) meter the buffer, or (v) any combination thereof.

In some embodiments, the motor 110 of the device 100 is operated, prior to the illuminating C), to rotate the disc 400 according to a second predefined profile to: (i) transfer one or more components of the biological sample 420 to a sample metering chamber 424 of the at least one slice, (ii) metering the one or more components of the biological sample 420, (iii) transfer the metered buffer from the buffer metering chamber 434 to a mixing chamber 436 of the at least one slice, or (iv) any combination thereof.

In some embodiments, the motor 110 of the device 100 is operated, prior to the illuminating C), to rotate the disc 400 according to a third predefined profile to: (i) transfer the metered one or more components of the biological sample 420 to the mixing chamber 436 of the at least one slice, (ii) promote mixing the metered one or more components of the biological sample 420 with the metered buffer, or (iii) both.

In some embodiments, the motor 110 of the device 100 is operated, prior to the illuminating C), to rotate the disc 400 according to a fourth predefined profile to: (i) transfer the fluid to one or more aliquoting chambers 440 of the at least one slice, (ii) divide the fluid into one or more aliquots, (iii) transfer an aliquot of the fluid to each of the one or more first wells 412 of the at least one slice, or (iv) any combination thereof.

In some embodiments, the motor 110 of the device 100 is operated, prior to the illuminating C), to rotate the disc 400 according to a fifth predefined profile to promote mixing of the aliquot of the fluid with the one or more reagents disposed in each of the one or more first wells 412.

In some embodiments, the motor 110 of the device 100 is operated, subsequent to the detecting D), to rotate the disc 400 according to a sixth predefined profile to: (i) transfer the aliquot of the fluid from each of the one or more first wells 412 to a second well 414, if present and in communication with the first well 412, of the at least one slice, (ii) promote mixing of the aliquot of the fluid with the one or more reagents disposed in the second well 414, or (iii) both.

In some embodiments, the motor 110 of the device 100 is operated, subsequent to the detecting D), to rotate the disc 400 according to the third predefined profile to transfer the aliquot of the fluid back to each of the one or more first wells 412.

Referring to block 926, in some embodiments, the method 900 includes L) repeating the illumination C) and detecting D). The repeating of repeating the illumination C) and detecting D) may be performed once or multiple times or as desired.

In some embodiments, the illumination C) and detecting D) are made for multiple time periods and the time-series measurement data (e.g., absorbance data) from each detector 124 is analyzed in the determining E) to ensure appropriate processes.

In some embodiments, the methods disclosed herein (e.g., the method 800 or 900) are performed by a controller 24. The controller 24 is configured for operating a device (e.g., the device 100 disclosed herein) for determining one or more analytes in a biological sample 420. The controller 24 includes one or more processors, and a memory coupled to the one or more processors. The memory includes one or more programs configured to be executed by the one or more processors, thereby causing the controller 24 to perform the methods disclosed herein. The controller 24 may be a component of the device 100 (e.g., embedded in the device 100), or may be external to the device 100. For instance, in some embodiments, the device 100 includes an interface or a controller 24, internal or external to the device 100, working alone or in combination with other controller 24(s). For instance, in some embodiments, the device 100 includes an electrical interface, to enable two-way communication with a master controller 24. Examples of such an electrical interface include but are not limited to an RS485 electrical interface.

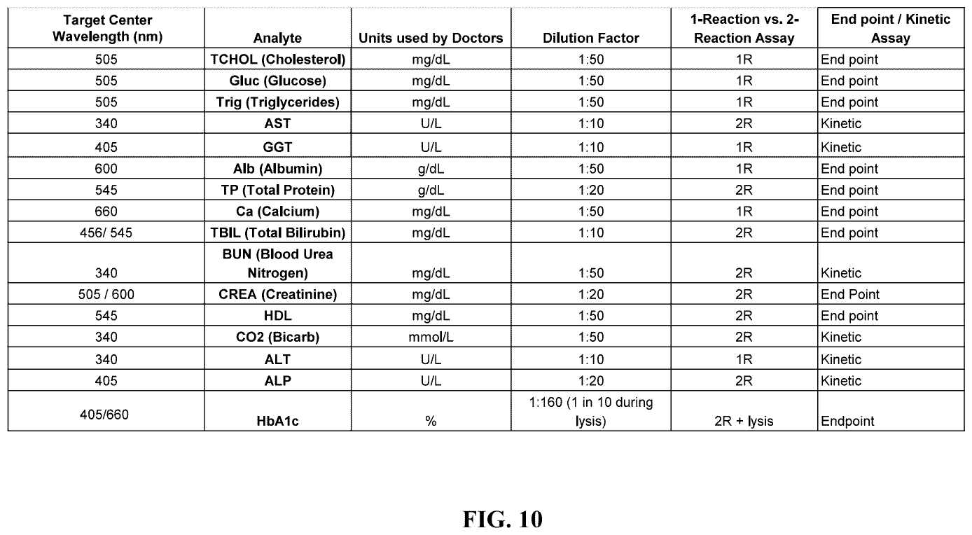

Referring to FIG. 10, there is shown in a table exemplary analytes that can be detected by the discs, devices and methods disclosed herein, and corresponding parameters that may be used in accordance with some exemplary embodiments of the present disclosure. In FIG. 10, the “1R” denotes a single-reaction assay, e.g., an assay with the reaction occurred in a first well 412 or with the reagent(s) disposed in the first well 412. The “2R” denotes a two-reaction assay, e.g., an assay with the reaction occurred in a first well 412 or with the reagent(s) disposed in the first well 412 as well as in a second or with the reagent(s) disposed in the second well 414. However, it should be noted that a single-reaction assay can be performed in a first well 412 (e.g., the first well 412-1 in FIG. 4A) that has a corresponding second well 414 (e.g., the second well 414-1 in FIG. 4A).

The discs, devices and methods of the present disclosure may be configured to include other additional, optional, or alternative features. For instance, in some embodiments, the absorbance data from at least one detector 124 or the temporal variation of the signal from at least one detector 124 is analyzed to determine if the measured signal is out of the pre-specified range, indicting the absence of a disc 400 or the presence of a dry disc 400.

In some embodiments, a non-reactive and spectrally non-interfering dye is doped in alternate reaction lyophilized beads. Absorbance level of this dye allows for determination of reaction volume as well as contamination between adjacent reaction wells (e.g., first wells 412 and second wells 414). For instance, in some embodiments, absorbance measurement of inert, spectrally non-interfering dye doped in the reagent lyophilized bead at a known concentration is used to establish accuracy of reaction volume, and presence of doped dye in a non-doped reaction well indicates contamination between reaction wells.

In some embodiments, discs, devices, and methods of the present disclosure are configured to perform reaction quality control. For instance, in some embodiments, the discs, devices and methods of the present disclosure are configured to allow for measuring standardized reactions, such as standardized chromogenic reactions, fluorescence-based assays, turbidity-based assays, or the like, to establish the viability of reagents in the standardized reactions. In some embodiments, a standardized reaction between a substrate and an enzyme is performed in at least one of the structures on the disc 400. This reaction leads to formation of chromophores at a known concentration. Absorbance measurements on this reaction are used to establish the validity of that particular detector 124 as well as reagents on the disc 400.

In some embodiments, the discs, devices, and methods of the present disclosure are configured to perform optical quality control. For instance, in some embodiments, the discs, devices and methods of the present disclosure are configured to allow for measuring absorbance for a standardized dye in one of the reaction wells to establish the performance of each optical detector. In some embodiments, a non-reactive dye mixture that absorbs at all relevant wavelengths (e.g., the wavelengths used by the device 100 for detecting the one or more analytes) is present in a lyophilized bead in a standardized amount. During the assay, this lyophilized bead is rehydrated in a well similar to the reaction well (e.g., the first well 412). Absorbance measurements on this well are used to establish the validity of the optical detectors.

The discs, devices, and methods of the present disclosure have a number of advantages. For instance, they allow for optical measurement while the disc 400 is spinning, with no need to stop the disc 400. This reduces the complexity in the device and operation. This also reduces the manufacturing cost. The discs, devices, and methods of the present disclosure also allow for multiple assays (e.g., 15-30 assays or more) on a single disc 400. This saves time and effort. Moreover, the discs, devices, and methods of the present disclosure allow for measurement and data acquisition for all chemical reactions, either kinetic reaction (e.g., measuring the slope) or endpoint reaction (e.g., waiting for the reaction to reach the plateau). A reaction usually occurs within a time period. Different reactions or assays may occur in very different time periods. For instance, one endpoint assay may take 3 minutes to run and the other may take 12 minutes to run. In many conventional settings, one has to synchronize all the chemistries to finish at the same time and then perform the measurement. In contrast, the discs, devices, and methods disclosed herein allow for measurement the whole time or any time as desired. Further, in some embodiments, the discs, devices, and methods of the present disclosure allow for measurement at multiple wavelengths and at every wavelength and every revolution while the disc 400 is spinning, and allow for spatial averaging (e.g., within a well) and temporal averaging (e.g., across multiple revolutions). The process is simple and robust. Furthermore, in some embodiments, the discs, devices and methods of the present disclosure allow for quality control such as abnormality detection or determination (e.g., leakage, cross contamination). In addition, the discs, devices, and methods of the present disclosure allow for cooling without the use of any powered components or devices, such as fans or the like, to create air circulation.

Some embodiments or implementations are described with respect to the following clauses:

Clause A1. A device for determining a plurality of analytes in a biological sample, the device comprising:
a motor configured to rotate a disc having a plurality of first wells at a first common radius relative to a rotational axis of the disc, wherein each of the plurality of first wells contains one or more reagents disposed therein and is configured to receive an aliquot of a fluid comprising one or more components of the biological sample;
a plurality of illumination sources configured to illuminate the plurality of first wells during a detection period while the motor is rotating the disc, wherein each of the plurality of illumination sources is disposed at the first common radius relative to the rotational axis of the disc and configured to provide illumination at a corresponding wavelength in a plurality of wavelengths; and
a plurality of detectors configured to detect one or more optical signals from the plurality of first wells during the detection period while the motor is rotating the disc, wherein each of the plurality of detectors is aligned with a corresponding illumination source in the plurality of illumination sources and configured to produce at least one reading for each of the plurality of first wells during the detection period, thereby facilitating determination of the plurality of analytes in the biological sample.
   Clause A2. The device of clause A1, wherein each of the plurality of detectors comprises a lens, a filter and a receiver.
   Clause A3. The device of clause A2, wherein the lens and filter are positioned at the first common radius relative to the rotational axis of the disc.
   Clause A4. The device of any one of clauses A1-A3, further comprising:
a first plate for housing the disc;
a first assembly disposed at a first side of the first plate and comprising the plurality of illumination sources;
a second assembly disposed at a second side of the first plate and comprising the plurality of detectors;
a third assembly disposed at or adjacent to the first plate and configured for regulating a temperature of the biological sample or the fluid comprising one or more components of the biological sample in the disc;
a fourth assembly disposed at or adjacent to the first plate and configured for determining a location of the disc relative to a home location;
an adapter plate configured for mounting the device to an instrument; or
any combination thereof.
   Clause A5. The device of clause A4, wherein the first, second, third or fourth assembly comprises a printed circuit board.
   Clause A6. The device of any one of clauses A4-A5, wherein the second assembly comprises a second plate disposed at a second side of the first plate and configured for mounting the plurality of detectors.
   Clause B1. A method for determining one or more analytes in a biological sample, the method comprising:
A) operating the motor of the device of any one of clauses A1-A6 to rotate a disc disposed in the device, wherein the disc comprises one or more first wells at a first common radius relative to a rotational axis of the disc, and each of the one or more first wells contains one or more reagents disposed therein and an aliquot of a fluid comprising one or more components of the biological sample;
B) illuminating, using the one or more illumination sources of the device, the one or more first wells during a detection period while the motor is rotating the disc;
C) detecting, using the one or more detectors of the device, one or more optical signals from the one or more first wells during the detection period while the motor is rotating the disc, wherein each of the one or more detectors produces at least one reading for each of the one or more first wells per revolution of the disc during the detection period; and
D) determining, based on the at least one reading for each of the one or more first wells per revolution of the disc during the detection period, the one or more analytes in the biological sample.
   Clause B2. The method of clause B1, wherein the operating A) rotates the disc during the detection period at a speed of at least about 500 revolutions per minute (rpm), at least about 600 rpm, at least about 700 rpm, at least about 800 rpm, at least about 900 rpm, at least about 1000 rpm, at least about 1200 rpm, at least about 1400 rpm, at least about 1600 rpm, at least about 1800 rpm, at least about 2000 rpm, at least about 2200 rpm, at least about 2400 rpm, at least about 2600 rpm, at least about 2800 rpm, at least about 2900 rpm, at least about 3000 rpm, at least about 3500 rpm, at least about 4000 rpm, at least about 4500 rpm, or at least about 5000 rpm.
   Clause B3. The method of any one of clause B1-B2, wherein:
an illumination beam of each of the one or more illumination sources is smaller than each of the one or more first wells;
in the detecting C), each of the one or more detectors produces a plurality of readings for each of the one or more first wells per revolution of the disc during the detection period; and
the determining D) comprises averaging the plurality of readings for each of the one or more first wells per revolution of the disc during the detection period.
   Clause B4. The method of clause B3, wherein the plurality of readings for each of the one or more first wells per revolution of the disc during the detection period comprises at least two readings, at least three readings, at least four readings, at least five readings, at least six readings, at least seven readings, at least eight readings, at least nine readings, at least ten readings, at least fifteen readings, or at least twenty readings.
   Clause B5. The method of any one of clauses B1-B4, wherein:
the detection period lasts for a plurality of revolutions; and
the determining D) comprises averaging the at least one reading for each of the one or more first wells per revolution of the disc during the detection period across at least a subset of the plurality of revolutions.
   Clause B6. The method of clause B5, wherein the subset of the plurality of revolutions has from two to ten revolutions, from three to twelve revolutions, or from four to fifteen revolutions.
   Clause B7. The method of clause B5, wherein the subset of the plurality of revolutions has three, four, five, six, seven, or eight revolutions.
   Clause B8. The method of any one of clauses B1-B7, wherein for each of the one or more first wells per revolution of the disc during the detection period, the detecting C) detects light transmitted through each of the one or more first wells, thereby producing the at least one reading that is indicative of light absorption by each of the one or more first wells.
   Clause B9. The method of clause B8, wherein the determining D) is based on Beer-Lambert's law.
   Clause B10. The method of any one of clauses B1-B9, wherein:
the detection period comprises a first detection period; and
the method further comprises: E) rotating, prior to the first detection period, the disc according to a first predefined profile to promote mixing of the aliquot of the fluid with the one or more reagents disposed in each of the one or more first wells.
   Clause B11. The method of clause B10, wherein the first predefined profile comprises alternately accelerating and decelerating rotation of the disc within a first speed range.
   Clause B12. The method of any one of clauses B10-B11, wherein:
the disc further comprises at least one second well positioned at a second common radius relative to the rotational axis of the disc, wherein the second common radius is shorter than the first common radius;
the detection period comprises a second detection period; and
the method further comprises: F) rotating, subsequent to the first detection period and prior to the second detection period, the disc according to a second predefined profile to promote mixing of the aliquot of the fluid with the one or more reagents disposed in each of the at least one second well.
   Clause B13. The method of clause B12, wherein the second predefined profile comprises alternately accelerating and decelerating rotation of the disc within a second speed range.
   Clause B14. The method of any one of clauses B1-B13, wherein:
the disc comprises a reference well at the first common radius relative to the rotational axis of the disc; and
the detecting C) comprises detecting, using the one or more detectors, one or more optical signals from the reference well during the detection period while the motor is rotating the disc, wherein each of the one or more detectors produces at least one reading for the reference well per revolution of the disc during the detection period, and wherein the at least one reading for the reference well is used as a reference.
   Clause B15. The method of clause B14, wherein for each of the one or more first wells, the determining D) comprises calculating one or more optical densities based on the at least one reading for the reference well and the at least one reading for each first well produced by each of the one or more detectors.
   Clause B16. The method of any one of clauses B1-B15, wherein the one or more first wells comprise a plurality of first wells spaced apart circumferentially from each other.
   Clause B17. The method of clause B16, wherein the detecting C) comprises detecting, using the one or more detectors, one or more optical signals from each spacing between adjacent first wells in the plurality of first wells during the detection period while the motor is rotating the disc, wherein each of the one or more detectors produces at least one reading for each spacing between adjacent first wells in the plurality of first wells per revolution of the disc during the detection period.
   Clause B18. The method of any one of clauses B16-B17, wherein:
the one or more illumination sources provide illumination at a plurality of wavelengths, wherein each of the plurality of wavelengths corresponds to one or more respective analytes; and
the one or more detectors comprises a plurality of detectors, each configured to detect an optical signal indicative of the one or more analytes at the corresponding wavelength.
   Clause B19. The method of clause B18, further comprising:
G) monitoring the at least one reading produced by each of the plurality of detectors for each of the plurality of first wells per revolution of the disc during the detection period; and
H) determining, based on the monitoring G), whether the at least one reading produced by each of the plurality of detectors for each of the plurality of first wells per revolution of the disc during the detection period is normal or abnormal.
   Clause B20. The method of clause B19, further comprising:
I) adjusting or terminating the method if it is determined that abnormality occurs.
   Clause B21. The method of clause B20, wherein the abnormality comprises leakage, cross contamination, or both.
   Clause C1. A device for determining one or more analytes in a biological sample, the device comprising:
a first plate configured for housing a disc having one or more first wells, wherein each of the one or more first wells contains one or more reagents disposed therein and is configured to receive an aliquot of a fluid comprising one or more components of the biological sample;
a radiation heat source disposed at or adjacent to the first plate and configured for maintaining the biological sample or the fluid comprising one or more components of the biological sample in the disc at a desired temperature; and
a motor configured to rotate the disc relative to the radiation heat source, wherein rotation of the disc creates air circulation between the disc and the radiation heat source, thereby facilitating uniform heating or cooling of the disc by the radiation heat source.
   Clause C2. The device of claim clause C1, wherein the radiation heat source is a part of a printed circuit board assembly disposed below the disc.
   Clause C3. The device of any one of clauses C1-C2, wherein the desired temperature is between 34° C. and 38° C., between 35° C. and 37° C., or about 36° C.
   Clause D1. A disc for determining one or more analytes in a biological sample, the disc comprising:
a rotational axis; and
one or more slices, each slice comprising:
   one or more first wells positioned at a first common radius relative to the rotational axis of the disc, wherein each of the one or more first wells contains one or more reagents disposed therein, and each of the one or more first wells is configured (i) to receive an aliquot of a fluid comprising one or more components of the biological sample, (ii) to allow the aliquot of the fluid mix with the one or more reagents disposed therein, and (iii) to facilitate optical detection of the one or more analytes in the biological sample.
      Clause D2. The disc of clause D1, further comprising:
a reference well at the first common radius relative to the rotational axis of the disc to serve as a reference.
   Clause D3. The disc of any one of clauses D1-D2, wherein the one or more slices comprise at least two, at least three, at least four, at least five, at least six, at least seven, at least eight, at least nine, or at least ten slices.
   Clause D4. The disc of clause D3, wherein some of the slices have a same number of first wells.
   Clause D5. The disc of any one of clauses D3-D4, wherein some of the slices have different numbers of first wells.
   Clause D6. The disc of any one of clauses D3-D5, wherein at least one slice comprises a plurality of first wells.
   Clause D7. The disc of any one of clauses D3-D6, wherein at least one slice comprises one or more second wells positioned at a second common radius relative to the rotational axis of the disc, each second well in fluidic communication with a corresponding first well.
   Clause D8. The disc of clause D7, wherein the second common radius is shorter than the first common radius.
   Clause D9. The disc of any one of clauses D7-D8, wherein the number of the one or more second wells is equal to or less than the number of the one or more first wells.
   Clause D10. The disc of any one of clauses D1-D9, wherein each slice further comprises:
a sample chamber positioned radially inward of the one or more first wells and configured for loading the biological sample.
   Clause D11. The disc of clause D10, wherein each slice further comprises:
a separation chamber in fluidic communication with the sample chamber and configured for separating the biological sample into multiple components.
   Clause D12. The disc of clause D11, wherein the biological sample is blood and one of the multiple components is plasma.
   Clause D13. The disc of any one of clauses D10-D12, wherein each slice further comprises:
a sample metering chamber in fluidic communication with the separation chamber and configured for metering one or more components of the biological sample.
   Clause D14. The disc of clause D13, wherein each slice further comprises:
a buffer chamber configured for loading a buffer;
a buffer metering chamber in fluidic communication with the buffer chamber and configured for metering the buffer; and
a mixing chamber in fluidic communication with the sample metering chamber and the buffer metering chamber and configured for mixing the metered one or more components of the biological sample with the metered buffer, thereby producing the fluid comprising the one or more components of the biological sample and the buffer at a dilution factor.
   Clause D15. The disc of clause D14, wherein the one or more slices comprise a first slice and a second slice.
   Clause D16. The disc of clause D15, wherein the buffer loaded to the first slice is the same as the buffer loaded to the second slice.
   Clause D17. The disc of clause D16, wherein the fluid in the first slice and the fluid in the second slice have a dilution factor.
   Clause D18. The disc of clause D16, wherein the fluid in the first slice and the fluid in the second slice have dilution factors.
   Clause D19. The disc of clause D15, wherein the buffer loaded to the first slice is different than the buffer loaded to the second slice.
   Clause D20. The disc of any one of clauses D14-D19, wherein each slice further comprises:
one or more aliquoting chambers in fluidic communication with the mixing chamber and configured for aliquoting the fluid into one or more aliquots,
wherein each of the one or more first wells is in fluidic communication with a corresponding aliquoting chamber to receive an aliquot of the fluid from the corresponding chamber.
   Clause D21. The disc of clause D15, wherein the one or more aliquoting chambers are positioned radially inward of the one or more first wells.
   Clause D22. The disc of any one of clauses D15-D21, wherein the one or more aliquoting chambers comprise a plurality of aliquoting chambers, wherein adjacent aliquoting chambers are connected with each other by a siphon structure.
   Clause E1. A device for determining one or more analytes in a biological sample, the device comprising:
a motor configured to rotate the disc of any one of clauses D1-D22;
one or more illumination sources configured to illuminate the one or more first wells during a detection period while the motor is rotating the disc; and
one or more detectors configured to detect one or more optical signals from the one or more first wells of each of the one or more slices during the detection period while the motor is rotating the disc, wherein each of the one or more detectors produces at least one reading for each of the one or more first wells of the one or more slices per revolution of the disc during the detection period, thereby facilitating determination of the one or more analytes in the biological sample.
   Clause E2. The device of clause E1, further comprising:
a first plate for housing the disc;
a first assembly disposed at a first side of the first plate and comprising the one or more illumination sources;
a second assembly disposed at a second side of the first plate and comprising the one or more detectors;
a third assembly disposed at or adjacent to the first plate and configured for regulating a temperature of the biological sample or the fluid comprising the one or more components of the biological sample in the disc;
a fourth assembly disposed at or adjacent to the first plate and configured for determining a location of the disc relative to a home location;
an adapter plate configured for mounting the device to an instrument; or
any combination thereof.
   Clause E3. A method for determining one or more analytes in a biological sample, the method comprising:
A) placing the disc of any one of clauses D1-D22 in the device of any one of clauses A1-A6;
B) operating the motor of the device to rotate the disc, wherein the one or more first wells of at least one slice in the one or more slices of the disc contain an aliquot of the fluid comprising one or more components of the biological sample;
C) illuminating, using the one or more illumination sources of the device, the one or more first wells of the at least one slice during a detection period while the motor is rotating the disc;
D) detecting, using the one or more detectors of the device, one or more optical signals from the one or more first wells of the at least one slice during the detection period while the motor is rotating the disc, wherein each of the one or more detectors produces at least one reading for each of the one or more first wells of the at least one slice per revolution of the disc during the detection period; and
E) determining, based on the at least one reading for each of the one or more first wells of the at least one slice per revolution of the disc during the detection period, the one or more analytes in the biological sample.
   Clause E4. The method of clause E3, wherein the biological sample comprises blood and the one or more components comprise plasma.
   Clause E5. The method of any one of clauses E3-E4, wherein the operating B) rotates the disc during the detection period at a speed of at least about 500 revolutions per minute (rpm), at least about 600 rpm, at least about 700 rpm, at least about 800 rpm, at least about 900 rpm, at least about 1000 rpm, at least about 1200 rpm, at least about 1400 rpm, at least about 1600 rpm, at least about 1800 rpm, at least about 2000 rpm, at least about 2200 rpm, at least about 2400 rpm, at least about 2600 rpm, at least about 2800 rpm, at least about 2900 rpm, at least about 3000 rpm, at least about 3500 rpm, at least about 4000 rpm, at least about 4500 rpm, or at least about 5000 rpm.
   Clause E6. The method of any one of clauses E3-E5, wherein:
an illumination beam of each of the one or more illumination sources is smaller than each of the one or more first wells of the at least one slice;
in the detecting D), each of the one or more detectors produces a plurality of readings for each of the one or more first wells of the at least one slice per revolution of the disc during the detection period; and
the determining E) comprises averaging the plurality of readings for each of the one or more first wells of the at least one slice per revolution of the disc during the detection period.
   Clause E7. The method of clause E6, wherein the plurality of readings for each of the one or more first wells per revolution of the disc during the detection period comprises at least two readings, at least three readings, at least four readings, at least five readings, at least six readings, at least seven readings, at least eight readings, at least nine readings, at least ten readings, at least fifteen readings, or at least twenty readings.
   Clause E8. The method of any one of clauses E3-E7, wherein:
the detection period lasts for a plurality of revolutions; and
the determining E) comprises averaging the at least one reading for each of the one or more first wells of the at least one slice per revolution of the disc during the detection period across at least a subset of the plurality of revolutions.
   Clause E9. The method of clause E8, wherein the subset of the plurality of revolutions has from two to ten revolutions, from three to twelve revolutions, or from four to fifteen revolutions.
   Clause E10. The method of clause E8, wherein the subset of the plurality of revolutions has three, four, five, six, seven, or eight revolutions.
   Clause E11, The method of any one of clauses E3-E10, wherein:
the disc comprises a reference well at the first common radius relative to the rotational axis of the disc; and
the detecting D) comprises detecting, using the one or more detectors, one or more optical signals from the reference well during the detection period while the motor is rotating the disc, wherein each of the one or more detectors produces at least one reading for the reference well per revolution of the disc during the detection period, and wherein the at least one reading for the reference well is used as a reference.
   Clause E12. The method of clause E11, wherein for each of the one or more first wells of the at least one slice, the determining E) comprises calculating one or more optical densities based on the at least one reading for the reference well and the at least one reading for each first well produced by each of the one or more detectors.
   Clause E13. The method of any one of clauses E3-E12, wherein the one or more first wells of the at least one slice comprise a plurality of first wells spaced apart circumferentially from each other.
   Clause E14. The method of clause E13, wherein the detecting D) comprises detecting, using the one or more detectors, one or more optical signals from each spacing between adjacent first wells in the plurality of first wells during the detection period while the motor is rotating the disc, wherein each of the one or more detectors produces at least one reading for each spacing between adjacent first wells in the plurality of first wells per revolution of the disc during the detection period.
   Clause E15. The method of any one of clauses E3-E14, wherein:
the one or more illumination sources provide illumination at a plurality of wavelengths, wherein each of the plurality of wavelengths corresponds to one or more respective analytes; and
the one or more detectors comprises a plurality of detectors, each configured to detect an optical signal indicative of the one or more respective analytes at the corresponding wavelength.
   Clause E16, The method of clause E15, further comprising:
F) monitoring the at least one reading produced by each of the plurality of detectors for each of the plurality of first wells per revolution of the disc during the detection period; and
G) determining, based on the monitoring F), whether the at least one reading produced by each of the plurality of detectors for each of the plurality of first wells per revolution of the disc during the detection period is normal or abnormal.
   Clause E17. The method of clause E16, further comprising:
H) adjusting or terminating the method if it is determined that abnormality occurs.
   Clause E18. The method of any one of clauses E3-E17, further comprising:
I) loading the biological sample into a sample chamber of at least one slice in the one or more slices of the disc;
J) loading a buffer into a buffer chamber of the at least one slice; or
both.
   Clause E19. The method of clause E18, wherein the loading I) loads the biological sample into the sample chamber of each of the one or more slices of the disc.
   Clause E20. The method of any one of clauses E18-E19, wherein the loading J) loads the buffer into the buffer chamber of each of the one or more slices of the disc.
   Clause E21. The method of any one of clauses E18-E20, wherein the loading I) or the loading J) is performed subsequent to the placing A).
   Clause E22. The method of any one of clauses E18-E21, further comprising:
K) operating, prior to the illuminating C), the motor of the device to rotate the disc according to one or more predefined profiles to:
   1) transfer the biological sample from the sample chamber to a separation chamber of the at least one slice;
   2) separate the biological sample into multiple components;
   3) transfer the buffer from the buffer chamber to a buffer metering chamber;
   4) meter the buffer;
   5) transfer one or more components of the biological sample to a sample metering chamber of the at least one slice;
   6) metering the one or more components of the biological sample;
   7) transfer the metered buffer from the buffer metering chamber to a mixing chamber of the at least one slice;
   8) transfer the metered one or more components of the biological sample to the mixing chamber of the at least one slice;
   9) promote mixing of the metered one or more components of the biological sample with the metered buffer, thereby producing the fluid comprising the one or more components of the biological sample and buffer at a defined dilution factor;
   10) transfer the fluid to one or more aliquoting chambers of the at least one slice;
   11) divide the fluid into one or more aliquots;
   12) transfer an aliquot of the fluid to each of the one or more first wells of the at least one slice;
   13) promote mixing of the aliquot of the fluid with the one or more reagents disposed in each of the one or more first wells; or
   any combination thereof.
      Clause E23. The method of clause E22, wherein the one or more predefined profiles comprise a first predefined profile to:
(i) transfer the biological sample from the sample chamber to a separation chamber of the at least one slice;
(ii) separate the biological sample into multiple components;
(iii) transfer the buffer from the buffer chamber to a buffer metering chamber;
(iv) meter the buffer; or
(v) any combination thereof.
   Clause E24. The method of any one of clauses E22-E23, wherein the one or more predefined profiles comprise a second predefined profile to:
(i) transfer one or more components of the biological sample to a sample metering chamber of the at least one slice;
(ii) metering the one or more components of the biological sample;
(iii) transfer the metered buffer from the buffer metering chamber to a mixing chamber of the at least one slice; or
(iv) any combination thereof.
   Clause E25. The method of any one of clauses E22-E24, the one or more predefined profiles comprise a third predefined profile to:
(i) transfer the metered one or more components of the biological sample to the mixing chamber of the at least one slice;
(ii) promote mixing the metered one or more components of the biological sample with the metered buffer; or
(iii) both.
   Clause E26. The method of any one of clauses E22-E25, wherein the one or more predefined profiles comprise a fourth predefined profile to:
(i) transfer the fluid to one or more aliquoting chambers of the at least one slice;
(ii) divide the fluid into one or more aliquots;
(iii) transfer an aliquot of the fluid to each of the one or more first wells of the at least one slice; or
(iv) any combination thereof.
   Clause E27. The method of any one of clauses E22-E26, wherein the one or more predefined profiles comprise a fifth predefined profile to promote mixing of the aliquot of the fluid with the one or more reagents disposed in each of the one or more first wells.
   Clause E28. The method of any one of clauses E22-E27, further comprising:
L) operating, subsequent to the detecting D), the motor of the device to rotate the disc according to a sixth predefined profile to:
   (i) transfer the aliquot of the fluid from each of the one or more first wells to a second well, if present and in communication with the first well, of the at least one slice;
   (ii) promote mixing of the aliquot of the fluid with the one or more reagents disposed in the second well; or
   (iii) both.
      Clause E29. The method of clause E28, further comprising:
M) operating, subsequent to the detecting D), the motor of the device to rotate the disc according to a third predefined profile to transfer the aliquot of the fluid back to each of the one or more first wells; and
N) repeating the illumination C) and detecting D).
   Clause F1. A controller for operating a device for determining one or more analytes in a biological sample, the controller comprising one or more processors, and a memory coupled to the one or more processors, the memory comprising one or more programs configured to be executed by the one or more processors, thereby causing the controller to perform the method of any one of clauses B1-B21 and E1-E29.
   Clause E2. The controller of clause F1, wherein the controller is a component of the device.
   Clause F3. The controller of clause F1, wherein the controller is external to the device.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will be understood that the terms “left” or “right”, “top” or “bottom”, “lower” or “upper”, “interior” or “exterior”, “inward” or “outward” and etc. are used to describe features of the exemplary embodiments with reference to the positions of such features as displayed in the figures. It will be understood that, although the terms “first,” “second,” etc. may be used herein to describe various elements, these elements should not be limited by these terms. These terms are only used to distinguish one element from another. For example, a first element could be termed a second element, and, similarly, a second element could be termed a first element, without changing the meaning of the description, so long as the “first element” and the “second element” are renamed consistently.

As used herein, the term “and/or” as used herein refers to and encompasses any and all possible combinations of one or more of the associated listed items. It will be further understood that the terms “include”, “includes”, “including”, “comprise”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

The term “if” used herein is, optionally, construed to mean “when” or “upon” or “in response to determining” or “in response to detecting” or “in accordance with a determination that,” depending on the context. Similarly, the phrase “if it is determined” or “if [a stated condition or event] is detected” used herein is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

When a reference number is given an “i^{th}” denotation, the reference number refers to a generic component, set, or embodiment. For instance, a “unit i” refers to the i^{th} unit in a plurality of units.

All references cited herein are incorporated herein by reference in their entirety and for all purposes to the same extent as if each individual publication or patent or patent application was specifically and individually indicated to be incorporated by reference in its entirety for all purposes.

What is claimed is:

1. A device for determining one or more analytes in a biological sample, the device comprising:
a motor configured to rotate a disc having one or more first wells at a first common radius relative to a rotational axis of the disc, wherein each of the one or more first wells contains one or more reagents disposed therein and is configured to receive an aliquot of a fluid comprising one or more components of the biological sample;
one or more illumination sources configured to illuminate the one or more first wells during a detection period while the motor is rotating the disc; and
one or more detectors configured to detect one or more optical signals from the one or more first wells during the detection period while the motor is rotating the disc, wherein each of the one or more detectors produces at least one reading for each of the one or more first wells per revolution of the disc during the detection period, thereby facilitating determination of the one or more analytes in the biological sample.

2.-3. (canceled)

4. The device of claim 1, wherein the one or more reagents comprise one or more lyophilized beads.

5.-13. (canceled)

14. The device of claim 1, wherein the disc further comprises at least one second well positioned at a second common radius relative to the rotational axis of the disc.

15.-17. (canceled)

18. The device of claim 1, wherein each of the one or more illumination sources is positioned at the first common radius relative to the rotational axis of the disc.

19. The device of claim 1, wherein for each of the one or more first wells, each of the one or more detectors produces a plurality of readings per revolution of the disc during the detection period.

20.-21. (canceled)

22. The device of claim 1, wherein each of the one or more detectors is positioned at the first common radius relative to the rotational axis of the disc.

23. The device of claim 1 further comprising:
a first plate for housing the disc;
a first assembly disposed at a first side of the first plate and comprising the plurality of illumination sources;
a second assembly disposed at a second side of the first plate and comprising the plurality of detectors;
a third assembly disposed at or adjacent to the first plate and configured for regulating a temperature of the biological sample or the fluid comprising the one or more components of the biological sample in the disc;
a fourth assembly disposed at or adjacent to the first plate and configured for determining a location of the disc relative to a home location;
an adapter plate configured for mounting the device to an instrument; or
any combination thereof.

24.-25. (canceled)

26. The device of claim 23, wherein:
the disc is rotatable relative to the third assembly; and
the third assembly comprises a radiation heat source configured for maintaining the biological sample or the fluid comprising the one or more components of the biological sample at a desired temperature.

27.-29. (canceled)

30. The device of claim 1, further comprising:
a spindle, wherein the disc is disposed on the spindle; and
a locking mechanism minimizing movement of the disc relative to the spindle, the locking mechanism comprising:
a locking element having a first end and a second end;
a collar extending radially from the first end; and
a disc lock disposed on the second end,
wherein the disc is received on the locking element between the collar and the disc lock.

31. A method for determining one or more analytes in a biological sample, the method comprising:
A) operating the motor of the device of claim 1 to rotate a disc disposed in the device, wherein the disc comprises one or more first wells at a first common radius relative to a rotational axis of the disc, and one or more of the first wells contains one or more reagents disposed therein and an aliquot of a fluid comprising one or more components of the biological sample;
B) illuminating, using the one or more illumination sources of the device, the one or more first wells during a detection period while the motor is rotating the disc;
C) detecting, using the one or more detectors of the device, one or more optical signals from the one or more first wells during the detection period while the motor is rotating the disc, wherein each of the one or more detectors produces at least one reading for each of the one or more first wells per revolution of the disc during the detection period; and
D) determining, based on the at least one reading for each of the one or more first wells per revolution of the disc during the detection period, the one or more analytes in the biological sample.

32. The method of claim 31, wherein:
in the detecting C), each of the one or more detectors produces a plurality of readings for each of the one or more first wells per revolution of the disc during the detection period; and
the determining D) comprises averaging the plurality of readings for each of the one or more first wells per revolution of the disc during the detection period.

33. The method of claim 31, wherein for each of the one or more first wells per revolution of the disc during the detection period, the detecting C) detects light transmitted through each of the one or more first wells, thereby producing the at least one reading that is indicative of light absorption by each of the one or more first wells.

34. The method of claim 31, wherein:
the detection period comprises a first detection period; and
the method further comprises: E) rotating, prior to the first detection period, the disc according to a first predefined profile to promote mixing of the aliquot of the fluid with the one or more reagents disposed in each of the one or more first wells.

35. The method of claim 34, wherein the first predefined profile comprises alternately accelerating and decelerating rotation of the disc within a first speed range.

36. The method of claim 34, wherein:
the disc further comprises at least one second well positioned at a second common radius relative to the rotational axis of the disc, wherein the second common radius is shorter than the first common radius;
the detection period comprises a second detection period; and
the method further comprises: F) rotating, subsequent to the first detection period and prior to the second detection period, the disc according to a second predefined profile to promote mixing of the aliquot of the fluid with the one or more reagents disposed in at least one second well.

37. The method of claim 36, wherein the second predefined profile comprises alternately accelerating and decelerating rotation of the disc within a second speed range.

38. (canceled)

39. The method of claim 31, wherein the one or more first wells comprise a plurality of first wells spaced apart circumferentially from each other, and wherein the detecting C) comprises detecting, using the one or more detectors, one or more optical signals from each spacing between adjacent first wells in the plurality of first wells during the detection period while the motor is rotating the disc, wherein each of the one or more detectors produces at least one reading for each spacing between adjacent first wells in the plurality of first wells per revolution of the disc during the detection period.

40. The method of claim 39, wherein:
the one or more illumination sources provide illumination at a plurality of wavelengths, wherein each of the plurality of wavelengths corresponds to one or more respective analytes; and
the one or more detectors comprises a plurality of detectors, each configured to detect an optical signal indicative of the one or more analytes at the corresponding wavelength.

41. The method of claim 40, further comprising:
G) monitoring the at least one reading produced by each of the plurality of detectors for each of the plurality of first wells per revolution of the disc during the detection period; and
H) determining, based on the monitoring G), whether the at least one reading produced by each of the plurality of detectors for each of the plurality of first wells per revolution of the disc during the detection period is normal or abnormal.

42. The method of claim 41, further comprising:
I) adjusting or terminating the method if it is determined that abnormality occurs.
